(19) United States
(12) Patent Application Publication
(10) Pub. No.: US 2024/0336959 A1
(43) Pub. Date: Oct. 10, 2024

(54) METHOD FOR MAKING A PHYSICAL MAP OF A POPULATION OF BARCODED PARTICLES

(71) Applicant: PIXELGEN TECHNOLOGIES AB, Solna (SE)

(72) Inventors: Simon FREDRIKSSON, Solna (SE); Filip KARLSSON, Solna (SE)

(21) Appl. No.: 18/034,595

(22) PCT Filed: Dec. 17, 2021
(86) PCT No.: PCT/IB2021/061892

§ 371 Date: Apr. 28, 2023

Related U.S. Application Data
(60) Provisional application No. 63/129,248, filed on Dec. 22, 2020.
(60) Provisional application No. 63/168,119, filed on Mar. 30, 2021.

(51) Int. Cl. C12Q 1/6841; C12Q 1/6806; C12Q 1/6874; C12Q 1/6876

(52) U.S. Cl. CPC C12Q 1/6841; C12Q 1/6806; C12Q 1/6874; C12Q 1/6876

(57) ABSTRACT

Provided herein is a method for making a physical map of a population of barcoded particles. In some embodiments, the method may involve: producing a complex comprising: i. a population of barcoded particles, wherein the barcoded particles are uniquely barcoded by surface-tethered oligonucleotides that have unique particle identifier sequences; and ii. a population of bridging moieties that comprises oligonucleotide sequences; wherein the bridging moieties are hybridized directly or indirectly to complementary sites in the surface-tethered oligonucleotides; performing a ligation, polymerization and/or a gap-fill/ligation reaction on the complex, thereby producing reaction products that comprise pairs of unique particle identifier sequences or complements thereof from adjacent barcoded particles: sequencing the reaction products, analyzing the sequences to making one or more physical maps of the barcoded particles. Systems for practicing the method are also provided.

### CROSS-REFERENCING

This application claims the benefit of U.S. provisional application Ser. Nos. 63/129,248, filed on Dec. 22, 2020, and 63/168,119, filed on Mar. 30, 2021, which applications are incorporated by reference herein.

### BACKGROUND

Cell polarity, i.e., the skewing of markers to one or more areas within or on the surface of a cell, is a common phenomenon but it is difficult to study in a high throughput way. For example, while there are several methods for analyzing the expression of cell surface markers on single cells (e.g., methods that involve flow cytometry or placing individual cells into compartments and then performing an assay on the individual cells), those methods do not provide any information about the spatial relationships of cell surface markers on the individual cells. More recent methods for analyzing the spatial relationships between biological molecules in or on cells, e.g., proximity ligation assays (see, e.g., Söderberg et al Nature Methods. 2006 3:995-1000), Weinstein's diffusion-based method (see, e.g., Cell 2019 178:229-241 and US20160265046), and array-based methods (see, e.g., Vickovic et al, Nature Methods 2019 16:987-990) are either not readily adapted to the analysis of cell surface markers or they do not provide any information about cell polarity. Microscopy is the gold-standard for analyzing spatial relationships between markers on single cells. However, microscopy is inherently very low throughput and challenging to automate.

In view of the above, a need still exists for methods analyzing cell polarity in a high throughput manner.

### SUMMARY

Provided herein is a method for making a physical map of a population of barcoded particles that, in some embodiments, may be attached to a surface, e.g., the surface of one or more cells. In some embodiments, the method may involve: producing a complex comprising: i. a population of barcoded particles, wherein the barcoded particles are uniquely barcoded by surface-tethered oligonucleotides that have unique particle identifier sequences and ii. a population of bridging moieties that comprises oligonucleotide sequences, wherein the bridging moieties are hybridized directly or indirectly to complementary sites in the surface-tethered oligonucleotides.

In this method, a ligation, polymerization and/or gap-fill/ligation reaction is performed on the complex, thereby producing reaction products that comprise pairs of unique particle identifier sequences from adjacent barcoded particles, or complements thereof. These reaction products are sequenced and the sequences are analyzed to identify which pairs of unique particle identifier sequences or complements thereof have been copied and/or ligated together. One or more physical maps of the barcoded particles can be made using the identified pairs of sequences. Systems for practicing the method are also provided.

This method may be used to analyze the distribution of markers that may be in or on a cell. In general, these embodiments may involve immobilizing particles in or on a target (e.g., a cell or a substrate), mapping the particles relative to one another, and then mapping the location and quantity of markers onto the particles via a proximity assay.

Certain aspects of this method are conceptually illustrated in FIGS. 2, 3, 7 and 8, although several variations are possible.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates a cell covered in particles.

FIG. 2 schematically illustrates a probe system comprising: i. a population of barcoded particles, wherein the barcoded particles (top) are uniquely barcoded by surface-tethered oligonucleotides that have unique particle identifier sequences (U1, U2, U3 . . . etc.), and ii. a population of bridging moieties that comprises oligonucleotide sequences (bottom), where the bridging moieties hybridize directly or indirectly via a splint to complementary sites in the surface-tethered oligonucleotides. In this figure, the surface-tethered oligonucleotides of the particles also contain B sequences that hybridize to complementary bridging moiety binding sequences (BMBS) sequences in the bridging moieties. As such, in this example, the bridging moieties hybridize directly to complementary sites in the surface-tethered oligonucleotides.

FIG. 3 schematically illustrates a complex between the barcoded particles and bridging moieties shown in FIG. 2 after hybridizing one to the other. A complex may contain at least a thousand barcoded particles, linked by bridging moieties.

FIG. 4 schematically illustrates the relative sizes of a barcoded particle (referred to as a UMI Clonal Bead in this figure), an antibody-oligonucleotide conjugate and a mammalian cell.

FIG. 5 schematically illustrates an example of how unique molecular identifiers from adjacent barcoded beads can be added to a bridging moiety. In this example: i. the complement of one of the unique molecular identifiers (e.g., UID2) can be added onto a 3′ end of the bridging moiety by extending the 3′ end of the bridging moiety using a surface-tethered oligonucleotide as a template and ii. the complement of the other of the unique molecular identifiers (e.g., UID1) can be added onto the 5′ end of a bridging by, e.g., by a gap-fill/ligation of an upstream oligonucleotide (as illustrated in FIG. 6 and described in greater detail below) or by ligation of the bridging moiety to an oligonucleotide that is complementary to the unique molecular identifier and hybridized to the surface-tethered oligonucleotide. In the latter embodiment, the surface-tethered oligonucleotide acts as a splint for ligation. In alternative embodiments, the bridging moieties can be ligated to the tethered oligonucleotides, e.g., using a splint.

FIG. 6 schematically illustrates one way by which UMIs from adjacent particles can be added to a bridging moiety by a gap-fill/ligation reaction. In practice, this implementation of the method may comprise producing a large complex such as that illustrated in FIG. 3, and adding polymerase, nucleotides and a ligase, thereby extending the ends of all bridging moieties that are hybridized to sequences in adjacent particles. Alternatively, the UMIs could be added by ligation.

FIG. 7 illustrates how the sequences at the ends of the extended bridging moieties, which contain sequences that identify the barcoded particle to which the original bridging moiety was bound, can be compared in a pairwise manner to provide a relational map of the barcoded particles.

FIG. 8 schematically illustrates a first implementation of the present method.

FIG. 9 schematically illustrates a second implementation of the present method.

FIG. 10 schematically illustrates the PCR amplicons produced using the method shown in FIG. 9.

FIG. 11 schematically illustrates a third implementation of the present method.

FIG. 12 schematically illustrates a fourth implementation of the present method.

FIG. 13 schematically illustrates a fifth implementation of the present method.

FIG. 14 schematically illustrates a sixth implementation of the present method.

FIG. 15 schematically illustrates a seventh implementation of the present method.

FIG. 16 schematically illustrates an eighth implementation of the present method.

FIG. 17 schematically illustrates the first part of a ninth implementation of the present method.

FIG. 18 schematically illustrates the second part of a ninth implementation of the present method.

FIG. 19 schematically illustrates a tenth implementation of the present method.

FIG. 20 schematically illustrates one way in which barcoded particles can be made using RCA products.

FIG. 21 schematically illustrates a related way in which barcoded particles can be made using RCA products.

FIG. 22 schematically illustrates an assay that uses barcoded particles as bridging moieties.

FIG. 23 schematically illustrates another way in which barcoded particles can be made using RCA products.

### DEFINITIONS

Before describing exemplary embodiments in greater detail, the following definitions are set forth to illustrate and define the meaning and scope of the terms used in the description.

Numeric ranges are inclusive of the numbers defining the range. Unless otherwise indicated, nucleic acids are written left to right in 5′ to 3′ orientation; and, amino acid sequences are written left to right in amino to carboxy orientation, respectively.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, thymine, uracil (G, C, A, T and U respectively). DNA and RNA have a deoxyribose and ribose sugar backbone, respectively, whereas PNA's backbone is composed of repeating N-(2-aminoethyl)-glycine units linked by peptide bonds. In PNA various purine and pyrimidine bases are linked to the backbone by methylene carbonyl bonds. A locked nucleic acid (LNA), often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. The term “unstructured nucleic acid”, or “UNA”, is a nucleic acid containing non-natural nucleotides that bind to each other with reduced stability. For example, an unstructured nucleic acid may contain a G′ residue and a C′ residue, where these residues correspond to non-naturally occurring forms, i.e., analogs, of G and C that base pair with each other with reduced stability, but retain an ability to base pair with naturally occurring C and G residues, respectively. Unstructured nucleic acid is described in US20050233340, which is incorporated by reference herein for disclosure of UNA.

The term “oligonucleotide” as used herein denotes a single-stranded multimer of nucleotides of from about 2 to 200 nucleotides, up to 500 nucleotides in length. Oligonucleotides may be synthetic or may be made enzymatically, and, in some embodiments, are 30 to 150 nucleotides in length. Oligonucleotides may contain ribonucleotide monomers (i.e., may be oligoribonucleotides) or deoxyribonucleotide monomers. An oligonucleotide may be 10 to 20, 21 to 30, 31 to 40, 41 to 50, 51to 60, 61 to 70, 71 to 80, 80 to 100, 100 to 150 or 150 to 200 nucleotides in length, for example.

The term “primer” as used herein refers to an oligonucleotide that is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence or fragment, the oligonucleotide primer typically contains 15-25 or more nucleotides, although it may contain fewer nucleotides. The primers herein are selected to be substantially complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

The term “hybridization” or “hybridizes” refers to a process in which a nucleic acid strand anneals to and forms a stable duplex, either a homoduplex or a heteroduplex, under normal hybridization conditions with a second complementary nucleic acid strand and does not form a stable duplex with unrelated nucleic acid molecules under the same normal hybridization conditions. The formation of a duplex is accomplished by annealing two complementary nucleic acid strands in a hybridization reaction. The hybridization reaction can be made to be highly specific by adjustment of the hybridization conditions (often referred to as hybridization stringency) under which the hybridization reaction takes place, such that hybridization between two nucleic acid strands will not form a stable duplex, e.g., a duplex that retains a region of double-strandedness under normal stringency conditions, unless the two nucleic acid strands contain a certain number of nucleotides in specific sequences which are substantially or completely complementary. “Normal hybridization or normal stringency conditions” are readily determined for any given hybridization reaction. See, for example, Ausubel et al., Current Protocols in Molecular Biology, John Wiley & Sons, Inc., New York, or Sambrook et al., Molecular Cloning: A Laboratory Manual, Cold Spring Harbor Laboratory Press. As used herein, the term “hybridizing” or “hybridization” refers to any process by which a strand of nucleic acid binds with a complementary strand through base pairing.

A nucleic acid is considered to be “selectively hybridizable” to a reference nucleic acid sequence if the two sequences specifically hybridize to one another under moderate to high stringency hybridization and wash conditions. Moderate and high stringency hybridization conditions are known (see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). One example of high stringency conditions includes hybridization at about 42 C in 50% formamide, 5×SSC, 5× Denhardt's solution, 0.5% SDS and 100 ug/ml denatured carrier DNA followed by washing two times in 2×SSC and 0.5% SDS at room temperature and two additional times in 0.1×SSC and 0.5% SDS at 42° C.

The term “sequencing”, as used herein, refers to a method by which the identity of at least 10 consecutive nucleotides (e.g., the identity of at least 20, at least 50, at least 100 or at least 200 or more consecutive nucleotides) of a polynucleotide are obtained.

The term “next-generation sequencing” refers to the so-called parallelized sequencing-by-synthesis or sequencing-by-ligation platforms currently employed by, e.g., Illumina, Life Technologies, BGI Genomics (Complete Genomics technology), and Roche etc. Next-generation sequencing methods may also include nanopore sequencing methods or electronic-detection based methods such as, e.g., Ion Torrent technology commercialized by Life Technologies.

The term “duplex,” or “duplexed,” as used herein, describes two complementary polynucleotides that are base-paired, i.e., hybridized together.

The terms “determining,” “measuring,” “evaluating,” “assessing,” “assaying,” and “analyzing” are used interchangeably herein to refer to forms of measurement, and include determining if an element is present or not. These terms include both quantitative and/or qualitative determinations. Assessing may be relative or absolute.

The term “ligating”, as used herein, refers to the enzymatically catalyzed joining of the terminal nucleotide at the 5′ end of a first DNA molecule to the terminal nucleotide at the 3′ end of a second DNA molecule.

A “gap-fill/ligation” reaction is a reaction in which two oligonucleotides are hybridized to a template with a gap in-between, one of the oligonucleotides is extended by a polymerase to fill in the gap, and the ligase seals the extended oligonucleotide to the other oligonucleotide.

A “polymerization” reaction is a reaction in which an oligonucleotide is hybridized to a template and the oligonucleotide is extended by a polymerase. There is no ligation to another oligonucleotide in a polymerization reaction.

The terms “plurality”, “set” and “population” are used interchangeably to refer to something that contains at least 2 members. In certain cases, a plurality may have at least 10, at least 100, at least 100, at least 10,000, or at least 100,000 members.

A “primer binding site” refers to a site to which an oligonucleotide hybridizes in a target polynucleotide or fragment. If an oligonucleotide “provides” a binding site for a primer, then the primer may hybridize to that oligonucleotide or its complement.

The term “strand” as used herein refers to a nucleic acid made up of nucleotides covalently linked together by covalent bonds, e.g., phosphodiester bonds.

The term “extending”, as used herein, refers to the extension of a primer by the addition of nucleotides using a polymerase. If a primer that is annealed to a nucleic acid is extended, the nucleic acid acts as a template for an extension reaction. Extending can also be done by ligation. For clarity: extending can be done by ligation, by a gap-fill ligation reaction, or by a polymerization reaction, as defined above.

As used herein, the term “surface” refers to any solid material (e.g. glass, metal, ceramics, organic polymer surface or gel) that may contains cells or any combinations of biomolecules derived from cells, such as proteins, nucleic acids, lipids, oligo/polysaccharides, biomolecule complexes, cellular organelles, cellular debris or excretions (exosomes, microvesicles), etc. Tissue blots, western blots and glass slides are examples of solid materials that have a surface. Cells, e.g., suspensions of mammalian cells, are another example of a surface.

As used herein, the term “splint” refers to an oligonucleotide that hybridize to the ends of two other oligonucleotides and brings those ends together to produce a ligatable junction.

As used herein, the term “population of barcoded particles” refers to particles, e.g., small beads or metallic particles the like, that are coated in oligonucleotides, where the surface-tethered oligonucleotides on each particle have a unique sequence that is different to the sequence that is in the oligonucleotides that are tethered to other particles in the population. In other words, if there are 1,000 barcoded particles, the oligonucleotides that are tethered to each particle will have a unique sequence (referred to herein as a unique molecular identifier “UMI” or unique identifier “UID”. The UID for one particle is different to the UIDs for other particles.

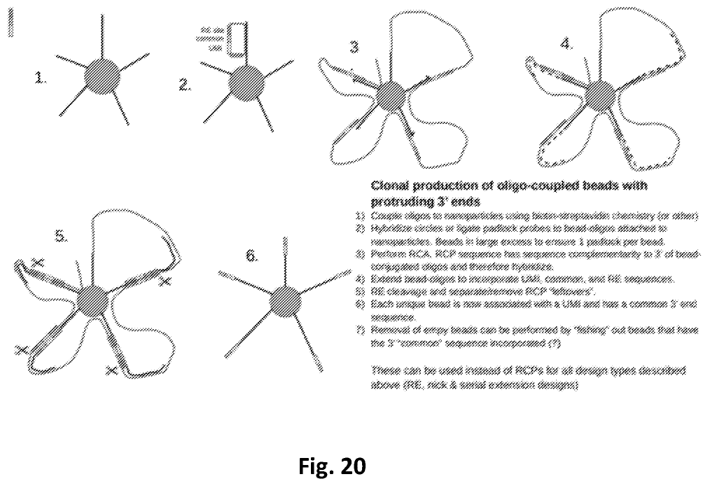

As used herein, the term “bridging moiety” refers to a moiety, that has at least two nucleic acid termini that can hybridize to other sequences and be extended by a polymerase or ligated onto by a ligase. Conventional oligonucleotides (which have 5′ and 3′ ends and are referred to herein as “bridging oligonucleotides”) are examples of bridging moiety, although other moieties could be used, e.g., oligonucleotides that have been directly or indirectly linked to one another. In some embodiment, a bridging moiety can be a particle that contains surface tethered oligonucleotides, as illustrated in FIGS. 20-22. As shown, these particles may have surface-tethered oligonucleotides that are barcoded, where the different particles have different barcodes.

As used herein, the term “hybridizing” refers to a reaction in which two sequence base pair with one another. Hybridizing requires at least 10 base pairs of complementarity between the sequences, although in many cases greater specificity can be obtained if there are at least 12, at least 15 base pairs of complementarity.

Other definitions of terms may appear throughout the specification.

### DESCRIPTION OF EXEMPLARY EMBODIMENTS

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

The following disclosure provides a way to map adjacent barcoded particles. The map produced by the method may be a three-dimensional map or a two-dimensional map, depending on how the method is implemented. For example, if the barcoded particles are immobilized in a three-dimensional way then the map produced may be three dimensional. In other embodiments, e.g., if the barcoded particles are immobilized on one or more surfaces (e.g., the surfaces of one or more cells that may be in suspension or mounted on a support), then the map produced by the method may be two-dimensional or three dimensional. In some embodiments, the method may result in multiple two-dimensional maps, where each map corresponds to the surface of a cell. While the method can be applied to cells (as described below) the method can be adapted to map adjacent barcoded particles that are immobilized on any surface, e.g., a glass slide that may have a tissue blot, or a western blot, etc. Likewise, although the barcoded particles or the bridging moieties to which the barcoded particles may be bound can be anchored to sites in a cell or on a surface via an antibody (e.g., an antibody that is conjugated to an oligonucleotide that has a sequence that is complementary to a sequence in the barcoded particles or bridging moieties), the barcoded particles or bridging moieties can be immobilized via using any type of interaction, e.g., covalent or non-covalent interactions, directly or indirectly, where in some embodiments the barcoded particles or bridging moieties may be bound to the cell via a non-antibody binding agent (e.g., an aptamer or an oligonucleotide, etc.), where the binding agent binds to a sequence in the barcoded particles or bridging moieties and a site that is in a cell or on the surface of the one or more cells. In some embodiments, the barcoded particles or bridging moieties may be immobilized via hybridization to an oligonucleotide that also hybridizes to a nucleic acid (e.g., to a cellular RNA). The barcoded particles or bridging moieties may be immobilized non-covalently to a site via an electrostatic interactions, via a streptavidin/biotin interaction, or by a covalent linkage (e.g., via a click coupling).

For the sake of clarity, the phrase “hybridizing the population of bridging moieties and the population of barcoded particles, wherein either the bridging moieties or the barcoded particles are immobilized” is intended to cover implementations where either: (a) the bridging moieties are hybridized to immobilized barcoded particles (in which case the barcoded particles are immobilized first, before the bridging moieties are hybridized) or (b) the barcoded particles are hybridized to immobilized bridging moieties (in which case the bridging moieties are immobilized or produced in situ first, before the barcoded particles are hybridized).

In any embodiment, the of bridging moieties or the population of barcoded particles molecules may be immobilized in or on cells that are in solution, cells that are one on a support (e.g., a slide), cells that in a three-dimensional sample of tissue, or cells that are in a tissue section. A sample containing cells that are in solution may be a sample of cultured cells that have been grown as a cell suspension, for example. In other embodiments, disassociated cells (which cells may have been produced by disassociating cultured cells or cells that are in a solid tissue, e.g., a soft tissue such as liver of spleen, using trypsin or the like) may be used. In particular embodiments, the population of bridging moieties or the population of barcoded particles may be immobilized on cells that can be found in blood, e.g., cells that in whole blood or a sub-population of cells thereof. Sub-populations of cells in whole blood include platelets, red blood cells (erythrocytes), platelets and white blood cells (i.e., peripheral blood leukocytes, which are made up of neutrophils, lymphocytes, eosinophils, basophils and monocytes). These five types of white blood cells can be further divided into two groups, granulocytes (which are also known as polymorphonuclear leukocytes and include neutrophils, eosinophils and basophils) and mononuclear leukocytes (which include monocytes and lymphocytes). Lymphocytes can be further divided into T cells, B cells and NK cells. Peripheral blood cells are found in the circulating pool of blood and not sequestered within the lymphatic system, spleen, liver, or bone marrow. If cells that are immobilized on a support are used, then then the sample may be made by, e.g., growing cells on a planar surface, depositing cells on a planar surface, e.g., by centrifugation, by cutting a three-dimensional object that contains cells into sections and mounting the sections onto a planar surface, i.e., producing a tissue section. In alternative embodiments, the surface may be made by absorbing cellular components onto a surface.

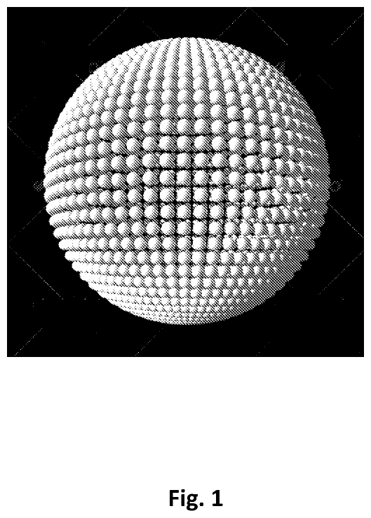

In any embodiment, the method may comprise immobilizing thousands, tens of thousands, hundreds of thousands, at least a million, at least 10 million, at least 100 million or at least a billion barcoded particles (each having a unique identifier), to a population of cells (e.g., via an antibody) so that barcoded particles coat the cells. Each cell may be coated in at least 100, at least 1,000 or at least 10,000 particles. A cell that is coated in particles is schematically illustrated in FIG. 1. As would be apparent, this figure is a schematic illustration: cells are not perfectly spherical and the barcoded particles may not be perfectly spherical, the same size or evenly distributed in a regular pattern, as shown. The barcoded particles may be anchored to the cells via an antibody or a nucleic acid probe although other methods are possible. In cases, the barcoded particles may be immobilized to the cell by hybridization to a bridging moiety. As will be described in greater detail below, each of the barcoded particles has a unique identifier sequence as well as a sequence to which a bridging moiety can hybridize. The bridging moieties and barcoded particles hybridize to produce a matrix comprising the barcoded particles and bridging moieties, where the bridging moieties are directly or indirectly hybridized to adjacent barcoded particles. After hybridization, the unique identifier sequences of adjacent barcoded particles are copied from the barcoded particles onto or ligated onto the bridging moieties. As will be described in greater detail below, the extended bridging moieties can be sequenced. A physical map of the barcoded particles can be constructed based on the sequences that have been added to the bridging moiety.

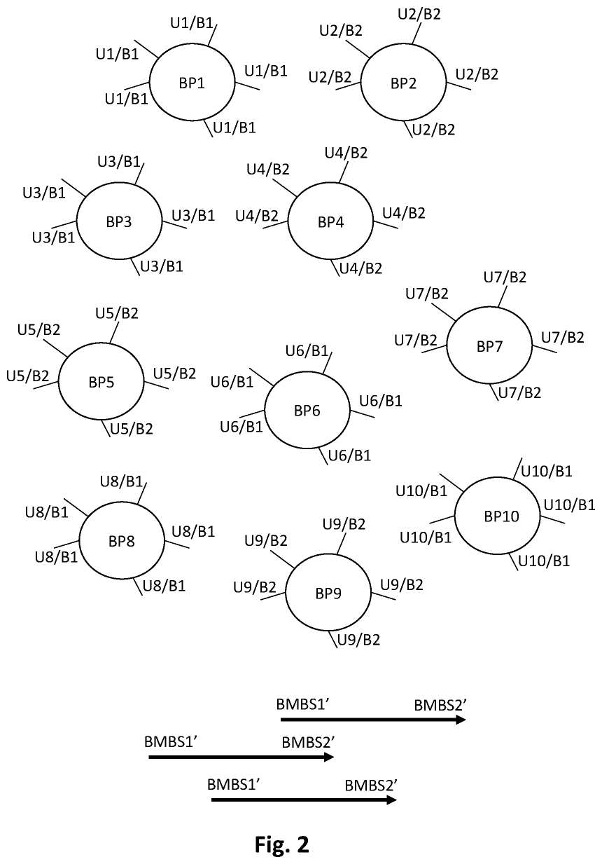

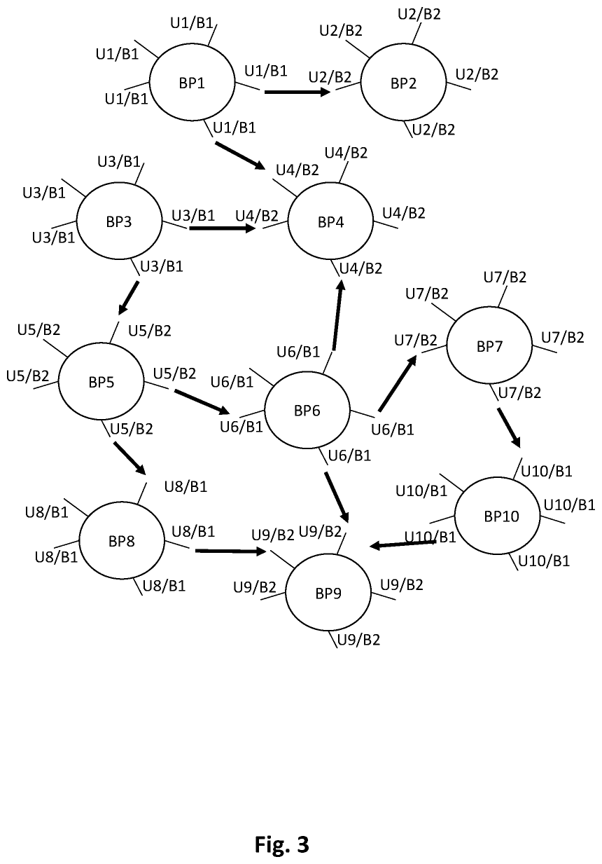

FIG. 2 shows a population of barcoded particles, wherein the barcoded particles are uniquely barcoded by surface-tethered oligonucleotides that have unique particle identifier sequences and a population of bridging moieties that comprises oligonucleotide sequences, wherein the bridging moieties hybridize directly or indirectly (e.g., via a splint) to complementary sites in the surface-tethered oligonucleotides of the barcoded particles. The population of barcoded particles shown in FIG. 2 is composed of 10 particles (BP1 to BP10). In practice, the number of barcoded particles used in the method may be much higher (e.g., in the millions or even billions), as noted above. In the example shown in FIG. 2, the barcoded particles are uniquely barcoded by surface-tethered oligonucleotides that have unique particle identifier sequences (UID1 to UID10), where UID is shortened to “U” in this figure. In the implementation shown in this figure, the surface-tethered oligonucleotides additionally contain a sequence that is complementary to a bridging moiety (a bridging moiety binding sequence or BMBS, which shortened to “B” in this figure). In this example, there are two types of BBBS in the barcoded particles, one type that has a BMBS1 sequence (shortened to “B1” in the figure) and the other type has another type that has a BMBS2 sequence (shortened to “B2” in the figure). The different BMBS sequences allow the bridging moieties (which in the embodiment shown have sequences that are complementary to BMBS1 and BMBS2) to preferentially hybridize to adjacent particles rather than to the same particle. Hybridization of the population of barcoded particles (shown at the bottom of FIG. 2) with the bridging moieties produces a complex comprising the population of barcode particles and the population of bridging moieties. This complex is schematically illustrated in FIG. 3. As will be described in greater detail below, the method can also be implemented without using different BBBS sequences.

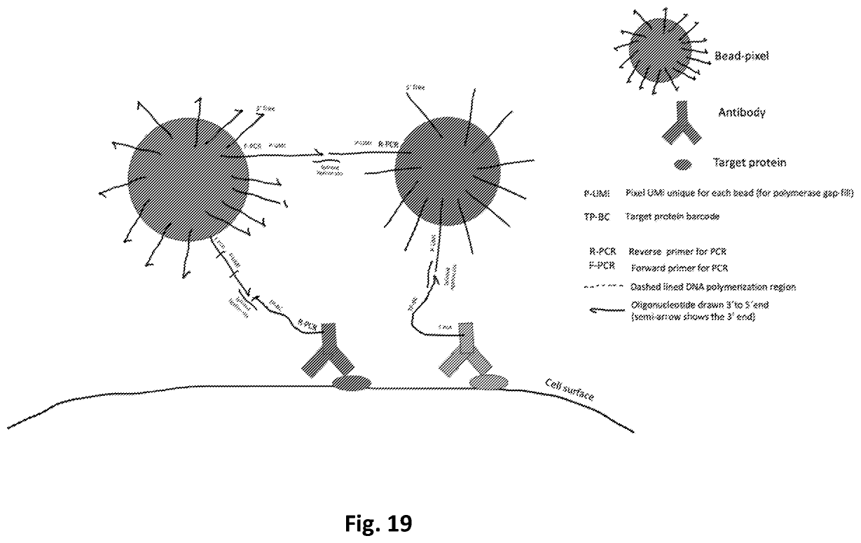

In FIGS. 2, 3, 5 and 6 and some of the subsequent figures, the bridging moiety is illustrated as an oligonucleotide that hybridizes to sequences that are in the surface-tethered oligonucleotides of the barcoded particles. As such, in some embodiments, the bridging moiety may be an oligonucleotide that has ends that are complementary to sequences in the surface-tethered oligonucleotides. In these embodiments, the surface-tethered oligonucleotides contain bridging moiety binding sequences, and the termini of the bridging moieties are complementary to those sequences and are capable of hybridizing thereto. In alternative embodiments, the bridging moieties may be hybridized indirectly to complementary sites in the surface-tethered oligonucleotides, e.g., via a splint. In these embodiments, the bridging moiety may be ligated to the surface-tethered oligonucleotides. In some cases, the bridging moiety could potentially contain two free 3′ ends, where the 3′ ends of the bridging moiety are complementary to and can hybridize to sequences that are at the distal end of the surface-tethered oligonucleotide. In these embodiments, the 3′ ends of the bridging moieties hybridize to the distal end of the surface-tethered oligonucleotides on adjacent barcoded particles and be extended to copy the UIDs from those surface-tethered oligonucleotides onto the to the bridging moiety. As such, in some embodiments, the bridging moieties may be splinted or unsplinted oligonucleotides that have ends that hybridize to or can be ligated to the surface tethered oligonucleotides in a splinted ligation reaction, or the bridging moieties may comprise oligonucleotide sequences and have free ends that can hybridize to the tethered oligonucleotides. In some embodiments and as illustrated in FIG. 19, the bridging oligonucleotides may act as a splint that joins the tethered oligonucleotides from one particle to another. In these embodiments, the tethered oligonucleotides in one type of particle should have free 5′ ends and the tethered oligonucleotides in one type of particle should have free 3′ ends.

The method may comprise hybridizing a bridging moiety (e.g., a population of grid oligonucleotide molecules or a population of particles that have surface-tethered oligonucleotides) and a population of barcoded particles, where the bridging moiety or the barcoded particles may be immobilized on one or more cells. This step can be implemented using a single type of bridging moiety (i.e., a population of the same molecule, having the same sequence), with an optional degenerate, e.g., random, sequence in the middle of molecule that could potentially serve as a molecule identifier. This method may be implemented using a population of barcoded particles that are otherwise the same except for the unique identifier sequence. However, in other embodiments the method may be implemented using two or more types of barcoded particles that differ in at least the unique particle identifier sequence as well as their bridging moiety binding sequence. This latter embodiment is illustrated in FIGS. 2 and 3. In the illustrated embodiment, the population of barcoded particles comprises: i. a first set of barcoded particles each comprising a unique particle identifier sequence (UID or “U”, as illustrated) and a first bridging moiety binding sequence (BMBS1 or “B1” as illustrated), and ii a second set of barcoded particles each comprising a unique particle identifier sequence (UID or “U”, as illustrated) and a second bridging moiety binding sequence (BMBS2 or “B2” as illustrated). There may be at least 1M, at least 10M, at least 100M or at least 1 B barcoded particles in the first set and a similar number (i.e., at least 1M, at least 10M, at least 100M or at least 1 B barcoded particles) in the second set, where each barcoded has a unique particle identifier sequence. As shown, in use, the first and second sets of barcoded particles are interspersed with each other such that a barcoded particle from the first set is likely to be proximal to at least one, but sometimes two, three or four barcoded particles from the second set.

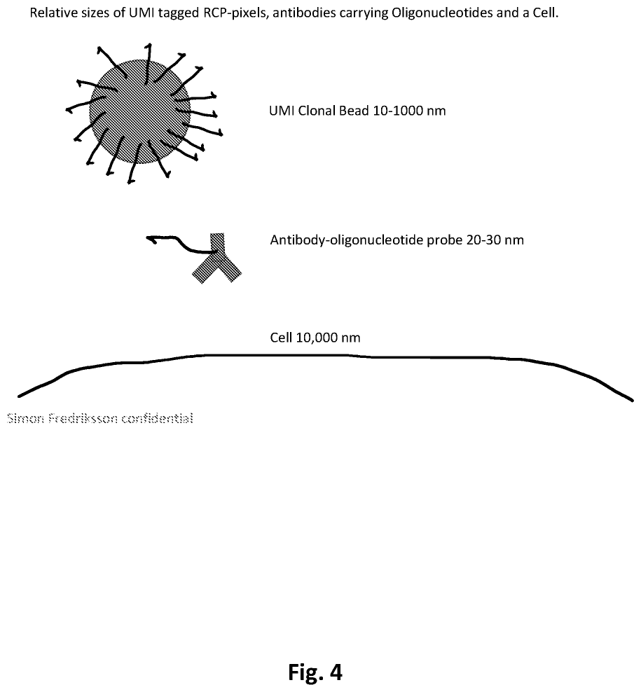

The particles can be of any suitable size, material and shape. FIG. 4 shows a particle relative to a mammalian cell. In many embodiments, the particles have size of 10-200 nm. The smaller the particle used, the higher the resolution of the image obtained. Gold particles (that can be readily made to any diameter in the range of 1.8 nm to 1500 nm, for example) can be used, although the particles can also be made from silver, silica, titanium dioxide, carbon, polymers (like polystyrene, polyacrylate, etc), agarose, etc. Magnetic particles of iron and various alloys could also be used (Creative Diagnostics, Shirley, NY, USA). Likewise, the particle can have any suitable shape, such as a sphere, rod, nanocube, plate, nanostar, etc. For example, spherical particles may be used (Creative Diagnostics, Shirley, NY, USA). The particles do not need to be magnetic, but magnetic nanospheres could be used in some cases (Creative Diagnostics, Shirley, NY, USA). There are several surface chemistries for functionalizing metal surfaces so that they can be joined to nucleic acid. For example, the particles may be modified to contain reactive groups, including, but not limited to, N-hydroxysuccinimidyl ester, sulfo-N-hydroxysuccinimidyl ester, a halo-substituted phenol ester, pentafluorophenol ester, a nitro-substituted phenol ester, an anhydride, isocyanate, isothiocyanate, an imidoester, maleimide, iodoacetyl, hydrazide, an aldehyde, or an epoxide. Other suitable groups are known in the art and may be described in, e.g., Hermanson, “Bioconjugate Techniques” Academic Press, 2nd Ed., 2008. The most commonly used capture-agent reactive groups are NHS (which is amine-reactive) and maleimide (which is sulfhydryl-reactive), although many others may be used. The particles can also be coated in streptavidin, which can bind to biotinylated nucleic acid. The barcoded particles described in this disclosure are not rolling circle amplification (RCA) products.

In some embodiments, the barcoded particles may be made by emulsion PCR, which method has been successfully used for other applications and is described in, e.g., Kanagal-Shamanna et al (Methods Mol Biol 2016 1392:33-42) and Shao et al (PlosOne 2011 0024910). In some embodiments, the method may involves coating a population of particles with a forward primer (e.g., via click chemistry, streptavidin, or via a covalent interaction), combining the particles with a reverse primer, dNTPs, polymerase and an oligonucleotide template that has a 5′ sequence that hybridizes with the forward primer, a variable, e.g., random, sequence that produces the UMIs when copied and a 3′ sequence corresponding to the reverse primer, producing an emulsion, where each droplet contains on average a single particle, a single molecule of template, and multiple molecules of reverse primers, and thermocycling the emulsion, thereby grafting copies of the sequence of the template onto the forward primers. The strand stranded that is not required can be subsequently removed by denaturation. Some aspects of emulsion PCR are described by Dressman et al. (PNAS 2003 100:8817-8822). In this example, streptavidin coated beads are bound to by a biotinylated PCR primer and combined with templates in a water and oil emulsion (plus the non-biotinylated second primer) to statistically only get I sequence copy per emulsion which is amplified on to the bead generating multiple copies of attached sequence. These bead bound sequences can be made single stranded by submerging the bead in sodium hydroxide and then washing (see Siu et al. Talanta 2021 221:121593). As would be understood, the template molecules may have a forward primer binding site, a degenerate (e.g., random) sequence of 6-10 nucleotides (or even more random nucleotides dependent on the number of unique particles required) and sequence that provides a binding site for the reverse primer, when it is copied.

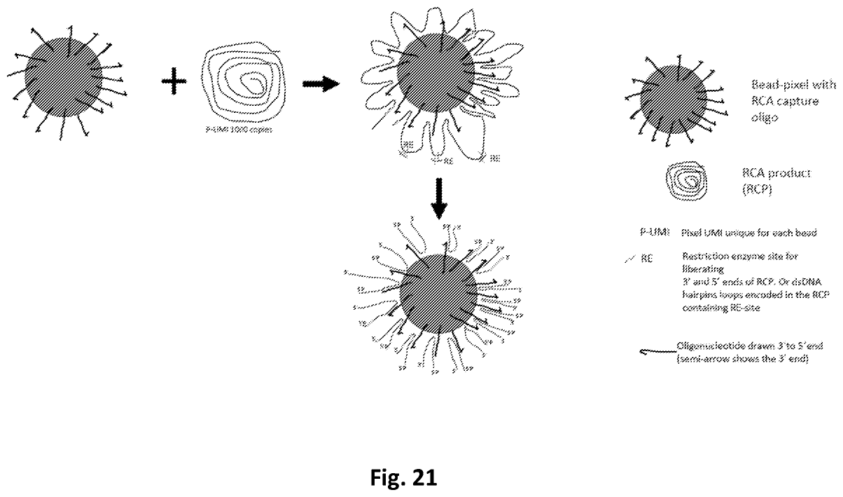

FIGS. 20 and 21 illustrate alternative ways by which barcoded particles can be made. As shown, barcoded particles may be made by combining an RCA product with a particle that contains surface-tethered primers, where the RCA product has a unique RCA identifier sequence and the primer hybridizes to a sequence in the RCA product that is upstream of the RCA identifier sequence. As is well known, “rolling circle amplification” or “RCA” for short refers to an isothermal amplification that generates linear concatemerized copies of a circular nucleic acid template using a strand-displacing polymerase. RCA is well known in the molecular biology arts and is described in a variety of publications including, but not limited to Lizardi et al (Nat. Genet. 1998 19:225-232), Schweitzer et al (Proc. Natl. Acad. Sci. 2000 97:10113-10119), Wiltshire et al (Clin. Chem. 2000 46:1990-1993) and Schweitzer et al (Curr. Opin. Biotech 2001 12:21-27), which are incorporated by reference herein. As used herein, the term “rolling circle amplification products” refers to the concatemerized products of a rolling circle amplification reaction. RCA products, which are using a circular molecule as a template using a strand-displacing DNA polymerase, are composed of a concatemer of hundred or thousands of copies of the reverse complement of the circle. As illustrated in FIG. 20, in this embodiment the repeated sequence of the RCA product may hybridize to the ends of multiple primer molecules on the particle, to a site that is upstream of a molecular identifier. Extension of the primer adds the reverse complement of the RCA identifier from the RCA product to the particle. Because each repeat of the RCA product contains the same identifier sequence, the same sequence will be added to each primer. As illustrated, the extension product, which is now double-stranded, can be cleaved by a restriction enzyme to produce a particle that contains multiple primers that each have the complement of a unique identifier, as copied from the same RCA product. An alternative method for producing particles that have surface tethered oligonucleotides that could be used in the present method is illustrated in FIG. 21. In this method, the barcoded RCA products are hybridized to the particles, and the RCA products are cleaved, thereby producing one or more ends that contain a unique identifier sequence.

These embodiments can be done by mixing particles that have surface-tethered primers with pre-made RCA products. Alternatively, as shown in FIG. 21, in some embodiments, the RCA reaction can be made “in situ”, i.e., by hybridizing a single circular template to each particle and extending one of the primer molecules that is tethered to the surface of the particle. As shown, this reaction should result in a concatemeric product that hybridizes to the other primers on the same particle. In some embodiments (and as illustrated) the RCA may be done “in situ”, i.e., using a primer that is tethered to the particle. In any embodiment, the hybridization reaction may be done at a sufficiently low dilution to avoid two circles (or RCA products) hybridizing to one particle. Alternatively, the RCA reactions or hybridizations can be done in compartments, e.g., in an emulsion, as described above.

Methods for generating circular molecules and RCA products that have unique identifiers are described in Wu et al (Nat. Comm. 2019 10:3854) and US20160281134, for example, and are readily adapted for use herein. In these embodiments, the circular molecules can be made by, e.g., synthesizing initial oligonucleotides that have a degenerate sequence, circularizing the initial oligonucleotides using a splint. If pre-made RCA products are used, the RCA products may be produced by amplifying the circularized oligonucleotides by RCA. In some embodiments, the initial oligonucleotides may contain a degenerate (e.g., random) sequence of 6-10 nucleotides, or even more random nucleotides dependent on the number of unique RCA products or circles required. The identifier sequence in the RCA products or circle can be 6-20 nucleotides in length, but identifier sequences that have a length outside of this range may be used in certain circumstances. In some embodiments, the sequence of the different RCA products or circles are the same except for the unique identifier sequence.

This embodiment of the method could be done by hybridizing at least 1,000 particles (e.g., at least 10,000, at least 100,000, at least a million, at least 10 million, at least 100 million or at least a billion particles), all having the same primer attached to their surfaces to suitable number of circles or RCA products that each have a unique identifier sequence (e.g., at least 1,000, at least 10,000, at least 100,000, at least a million, at least 10 million, at least 100 million or at least a billion circles or RCA products that have a unique identifier sequence). In this method, the unique identifier sequences are transferred from the RCA products to the particles, thereby producing a population of barcoded particles that can be used in the present method.

A method for manufacturing barcoded particles is therefore provided. These methods are generally described in FIGS. 20 and 21 and may comprise: (a) hybridizing a population of RCA products that have unique identifier sequences with a population of particles that have surface-tethered oligonucleotides, wherein a plurality of the particles hybridize to a single RCA product and multiple surface-tethered oligonucleotides of each of the plurality of the particles hybridize to a site in the RCA product that is upstream of the identifier sequences, and (b) either cleaving the RCA products at a site that is downstream of the identifier sequences in the RCA products, or extending the surface tethered oligonucleotides using the hybridized RCA products as a template, thereby adding identifier sequences from the RCA products to the particles. As noted above, this latter embodiment can be done by hybridizing pre-made RCA products with the particles or RCA products that are made in situ, i.e., on the particles, as illustrated in FIG. 21.

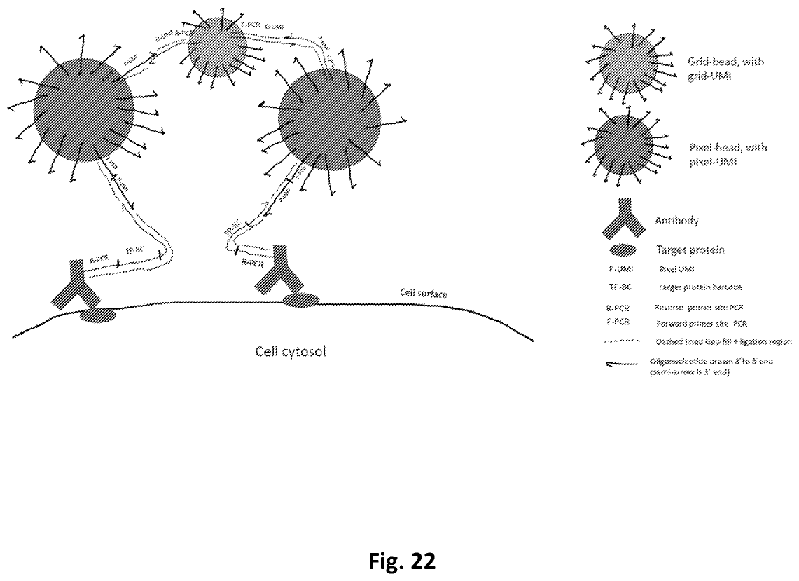

An example of an assay of that uses such particles as bridging moieties is shown in FIG. 22.

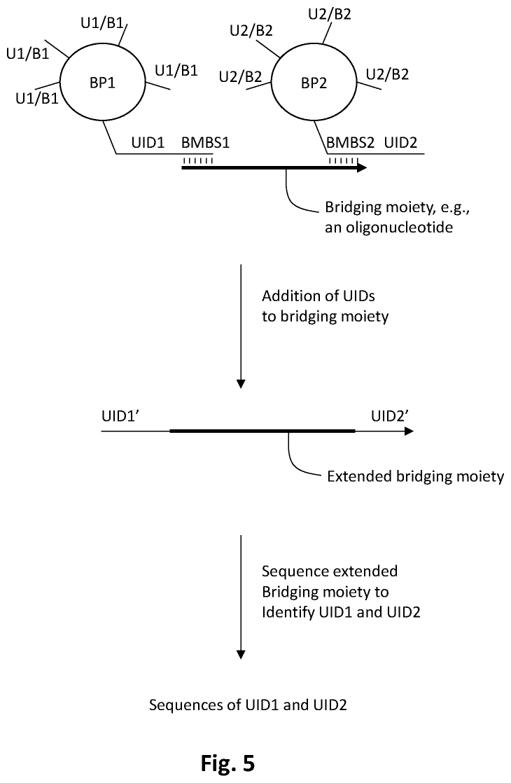

As shown in FIG. 5, in some embodiments the bridging moieties used in the method can each comprise a first terminal sequence that is complementary to the first bridging moiety binding sequence (BMBS1 or B1) and a second terminal sequence that is complementary to the second bridging moiety binding sequence (BMBS2 or B2). In this example, at least some of the bridging moieties hybridize to two adjacent barcoded particles via those sequences. FIG. 5 shows a bridging moiety that is hybridized to two adjacent barcoded particles. Assuming that consecutive bases are distanced by approximately 0.3 nm, then a 100-mer oligonucleotide should, in theory, be able to stretch 30 nm, a 200-mer bridging moiety that is an oligonucleotide should, in theory, be able to stretch 60 nm and a 500-mer bridging moiety should, in theory, be able to stretch 150 nm. As such, the barcoded particles to which a bridging moiety hybridizes may be less than 100 nm apart or less than 50 nm apart. As would be apparent, particles that are closer together provide a higher resolution. In some embodiments, e.g., embodiments in which the bridging moiety acts as a ligation splint to facilitate ligation of the particles to one another (see, e.g., FIG. 19), the bridging moiety may be a relatively short oligonucleotide (e.g., an oligonucleotide in the range of 12-30 nt).

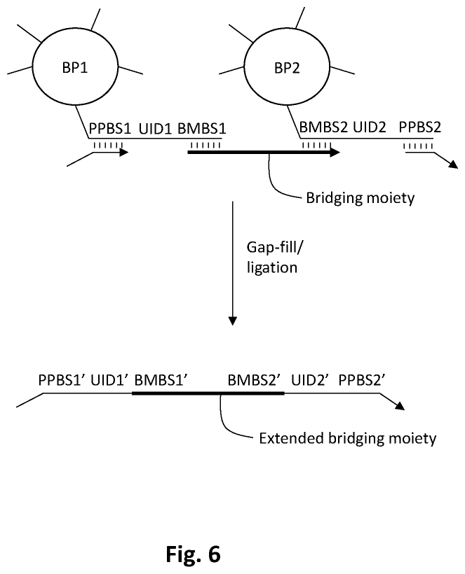

In the next step of the method, the bridging moieties that are hybridized to two adjacent particles can be extended to add the unique particle identifier sequences from two adjacent particles or their complements to ends of the bridging moieties, thereby producing extended bridging moieties. In the example shown in FIG. 5, UID1′ (i.e., the complement of UID1) is added to one end of the bridging moiety and UID2′ (the complement of UID2) is added to the other end of the bridging moiety. As shown in FIG. 6, in some embodiments, the bridging moiety may be extended using a gap fill/ligation reaction (see, e.g., Mignardi et al, Nucleic Acids Res. 2015 43: e151) that adds complements of the unique particle identifier sequences from the two adjacent particles to the bridging moiety.

Hybridization of these components together can produce a complex in which the first and second unique particle identifier sequences can be copied by extending the 3′ and 5′ ends of the bridging moiety, respectively, via a gap-fill/ligation reaction.

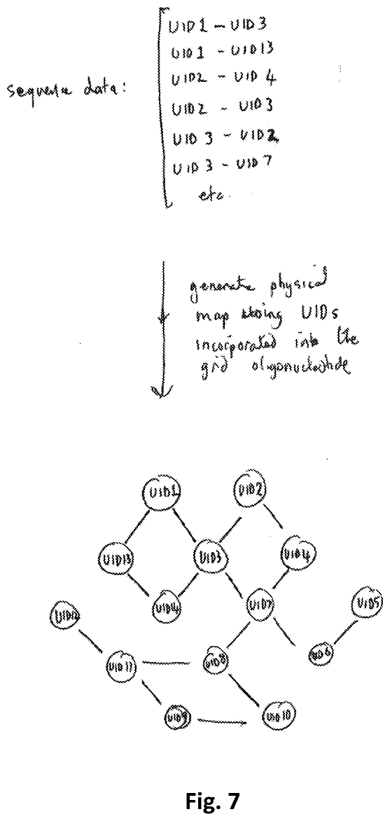

As illustrated in FIG. 5, after the bridging moieties have been extended to add the UIDs from adjacent barcoded particles onto their ends, the extended bridging moieties are sequenced and then analyzed to identify which pairs of unique particle identifier sequence complements have been added onto the bridging moieties. This process is illustrated in FIG. 7. As illustrated in FIG. 7, each extended bridging moieties should have the complement of a first unique particle identifier sequence at one end (e.g., UID1) and the complement of a first unique particle identifier sequence at the other (e.g., UID3). These sequences can be analyzed to compile a list of paired particle identifier sequences (e.g., UID1-UID3, UID1-UID13, etc.) which can be used to make a two-or three-dimensional map of the particles. As illustrated in FIG. 7, the method may involve making one or more physical maps (a relational map) of the immobilized particles using the list paired particle identifier sequence of sequences. As would be apparent, the map may be a map of the surface of one or more cells. In some cases, the physical maps may comprise overlapping and/or non-overlapping maps.

FIG. 6 shows one way in which the UMIs may be added to the bridging moieties. In this example, the reaction that involves: (a) producing a complex of the bridging moieties and barcoded particles as illustrated in FIG. 2, where, in the complex, the end sequences of the bridging moiety (e.g., 3′ and 5′ end sequences of an oligonucleotide) are hybridized to the first and second bridging moiety binding sequences (BMBS1 and BMBS2) of the first and second barcoded particles, respectively; and (b) treating the complex of (a) with a polymerase and a ligase, thereby copying (via a gap-fill/ligation reaction) the complements of the first and second unique particle identifier sequences onto the 3′ ends of the bridging moiety thereby producing a product molecule (an extended bridging molecule) that comprises the complements of the first and second unique particle identifier sequences. FIG. 6 shows how this reaction can be done using upstream and downstream primers, which, as noted below, may be non-covalently tethered to sites on a surface. As would be apparent, if the bridging moieties have two 3′ ends, the ends can hybridize to the barcoded parties and the particle identifier sequences can be copied onto those ends via a primer extension reaction. Either way, the complements of pairs of unique sequences that identify adjacent particles are copied into a bridging moiety.

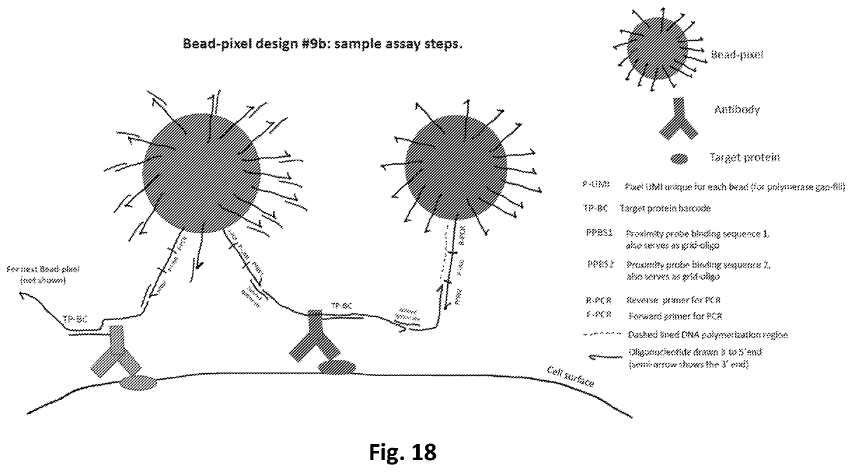

In other embodiments, the addition may be done by ligation as mentioned above and, in some embodiments, the surface-tethered oligonucleotides may be ligated together. In these embodiments, the bridging moiety acts as a splint (as shown in FIG. 19). Likewise, the addition may be done by polymerization, as illustrated in FIG. 18.

In any embodiment, the extended bridging moieties and other molecules that have been extended in the assay may be amplified by PCR prior to sequencing. Depending on the method being used, this may be done using one, two, three or four primer pairs (which may be done in a multiplexed reaction). In some of these embodiments, the binding sites for the PCR primers may be added to the 3′ and 5′ tails of the upstream and downstream primers respectively, as illustrated in FIG. 6 or, in theory, the binding sites for the PCR primers could be coded into the oligonucleotides that are tethered to the particles and copied onto the ends of the bridging moieties during the extension reaction.

In addition to making a map of the barcoded particles, the method may involve performing a proximity assay between one or more binding agents that are bound to sites in cells or on the surface of cells (e.g., antibodies that are bound to cell surface markers on the cells). In these embodiments, a unique particle identifier sequence may be copied into an oligonucleotide that is linked to the capture agent. In some embodiments, the capture agent is an antibody-oligonucleotide conjugate and in other embodiments, the capture agent may be an oligonucleotide probe. In these embodiments, the terms “antibody-oligonucleotide conjugate” and “capture agent that is linked to an oligonucleotide” refers to a capture agent, e.g., an antibody or aptamer, that is non-covalently (e.g., via a streptavidin/biotin interaction) or covalently (e.g., via a click reaction or the like) linked to a single-stranded oligonucleotide in a way that the capture agent can still bind to its binding site. The oligonucleotide and the capture agent may be linked via a number of different methods, including those that use maleimide or halogen-containing group, which are cysteine-reactive. The capture agent and the oligonucleotide may be linked proximal to or at the 5′ end of the oligonucleotide, proximal to or at the 3′ end of the oligonucleotide, or anywhere in-between. In some embodiments, the oligonucleotides may be linked to the capture agents by a linker that spaces the oligonucleotide from the capture agents. Oligonucleotides may be linked to capture agents using any convenient method (see, e.g., Gong et al., Bioconjugate Chem. 2016 27:217-225 and Kazane et al. Proc Natl Acad Sci 2012 109:3731-3736). In many embodiments, the sequence of an oligonucleotide that is conjugated to a binding agent uniquely identifies the epitope or sequence to which the binding agent binds. For example, if the method is performed using 10 different antibodies, then each antibody is tethered to a different sequence that identifies the epitope to which the antibody binds. This feature allows the method to be multiplexed and, in some embodiments, at least 5, at least 10, at least 20 or at least 50 different antibodies that bind to different markers in or on the surface of a cell can be used in the method. Each antibody is conjugated to a different antibody identifier sequence, and the antibody identifier sequences allow the binding events for a particular antibody to be mapped. Such tagged antibodies are described in, e.g., Wu et al (Nat. Comm. 2019 10:3854) and US20160281134, and others.

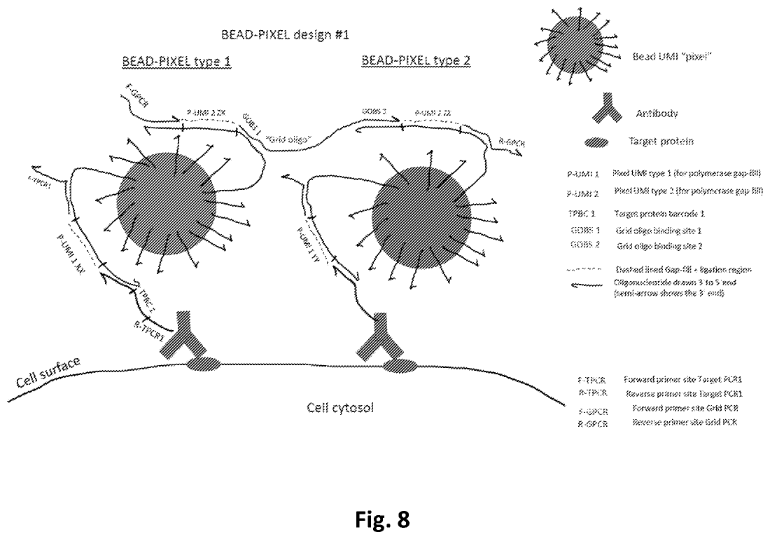

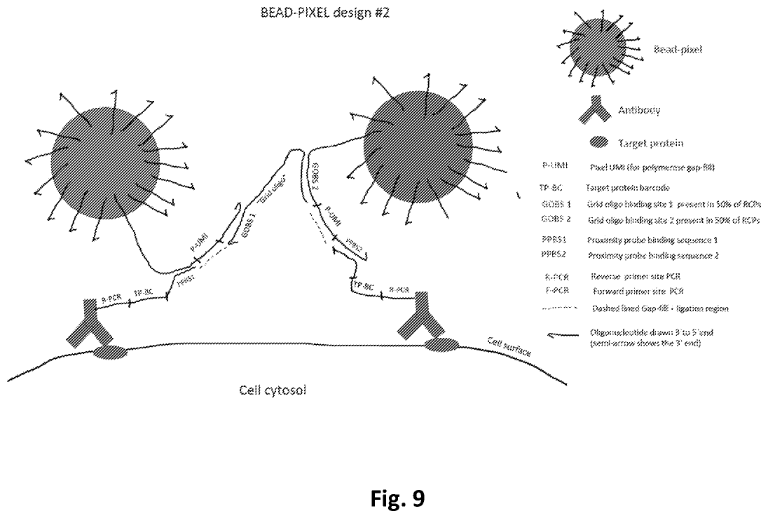

As illustrated in FIGS. 8-18, the proximity assay may be implemented in a variety of different ways. In any embodiment, the proximity assay may produce products that contains a binding agent identifier sequence or complement thereof and a unique particle identifier sequence or complement thereof. In some embodiments, the product of the proximity assay (an extended proximity probe) may be a separate molecule to the extended bridging moiety. In other embodiments, the product of the proximity assay (an extended proximity probe) may be part of the extended bridging moiety (see, e.g., FIG. 9). In any embodiment, a portion of the capture agents used in the assay may be linked to the 5′ ends of the oligonucleotides and the remainder of the capture agents may be linked to the 3′ ends of the oligonucleotides. For example, in some embodiments, the method may make use of a mixture comprising one or more antibodies-oligonucleotide conjugates, where in some embodiments, some of the antibodies that bind to a particular cell surface marker (e.g., 30%-70% of the antibody molecules) are conjugated to the 5′ end of an oligonucleotide and the remainder of the antibodies that bind to that cell surface marker are conjugated to the 3′ and of an oligonucleotide. In these embodiments, the oligonucleotides may each contain a PCR primer binding site (at whichever end of the oligonucleotide that is linked to the antibody) and the product produced by the assay may be amplifiable by PCR.

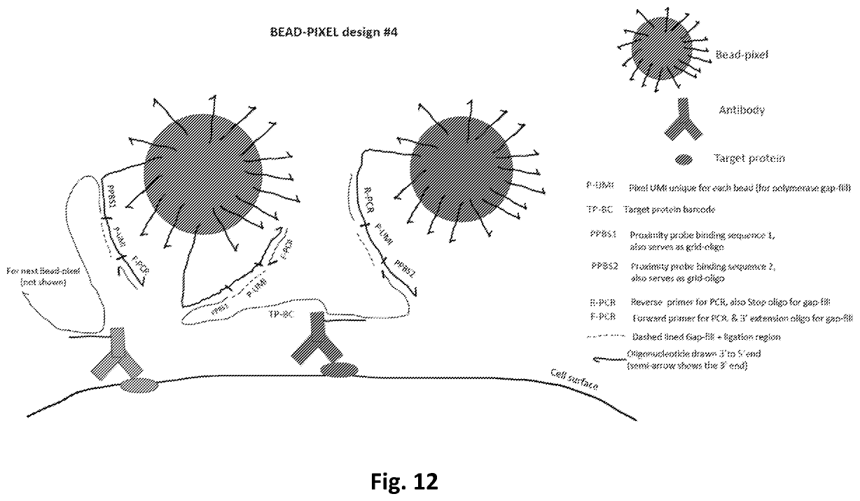

As illustrated in FIG. 12, in some embodiments, the bridging moieties may be immobilized to the surface of a cell prior to the addition of the barcoded particles. In these embodiments, the method may comprise (a) hybridizing a population of barcoded particles with a population of bridging moieties that are immobilized on one or more surfaces, wherein: (i) the barcoded particles of the population of barcoded particles each have a unique particle identifier sequence and a bridging moiety binding sequence, and (ii) the bridging moieties each comprise a first terminal sequence that is complementary to a bridging moiety binding sequence and a second terminal sequence that is complementary to a bridging moiety binding sequence; and (iii) at least some of the bridging moieties hybridize to two adjacent particles; (b) extending the bridging moieties that are hybridized to two adjacent particles to add the unique particle identifier sequences from two adjacent particles, or the complements thereof, to the bridging moieties, thereby producing extended bridging moieties; (c) sequencing the extended bridging moieties; and (d) analyzing the sequences to identify which pairs of unique particle identifier sequence complements have been added onto the bridging moieties. In some embodiments, the bridging moiety may, itself, be the product of a proximity ligation assay. In these embodiments, the bridging moieties may be split such that each portion hybridizes to a different probe. In these embodiments, an intact bridging moiety is only produced if the two parts of the bridging moiety are proximal to each other and capable of ligating to one another in a splinted ligation reaction.

The method may comprise making a physical map of the immobilized particles using the pairs of unique particle identifier sequences identified by analysis of the sequence reads and, also, mapping the binding agents to the physical map of the immobilized particles by analyzing which unique particle identifier sequences and which binding agent identifier sequences are in the assay products. In some embodiments, the complement of a binding agent identifier sequence and the complement of a unique particle identifier sequence can be incorporated into the extended bridging moiety. In other embodiments, the complement of a binding agent identifier sequence and the complement of a particle product identifier sequence are incorporated into assay products that are separate from the extended bridging moieties. Analysis of the unique particle identifier sequences that are copied into the assay products in the proximity assay allows the binding sites for each of the capture agents that is bound to the cell to be mapped to a particular particle. Specifically, each binding event can be mapped to a particle because the unique particle identifier sequence for that particle are added to binding agent-tethered oligonucleotides that are proximal to that particle. The binding agents can then be placed on the map of particles described above, thereby providing a two-or three-dimensional map of the binding events, where the map may correspond to the surfaces of one or more cells.

As would be apparent, each barcoded particle contains multiple copies of the same sequence and, as such, multiple binding events can be mapped to a barcoded particle, thereby providing a way to quantify the particles. For example, if a hundred antibody-oligonucleotide conjugates bind to sites that are all proximal to a particular particle, then all hundred binding sites can potentially be mapped to a particle. Mapping binding sites to particles that, themselves, have been mapped in two dimensions provides a way to examine the distribution of binding sites in or on the surface of a cell. This, in turn, provides a way to examine cell polarity without microscopy.

Also provided herein is a probe system. As illustrated in FIG. 2, in some embodiments, the probe system may comprise: (a) a population of barcoded particles, wherein the barcoded particles are uniquely barcoded by surface-tethered oligonucleotides that have a unique particle identifier sequence and a bridging moiety binding sequence; and (b) a population of bridging moieties that comprises oligonucleotide sequences, wherein the oligonucleotide sequences are complementary to the bridging moiety sequence. As noted above, hybridization of (a) and (b) produces a complex in which the bridging moiety hybridize to adjacent particles, as shown in FIG. 3. The population of barcoded particles may comprise at least 10 members (e.g., at least 100, at least 1,000, at least 1,000, at least 10,000, at least 100,000, at least IM at least 10 M, at least 100 M, at least 1 B or at least 10 B) members. In some embodiments, the bridging moiety binding sequences in the particles are adjacent to the unique particle identifier sequences in the oligonucleotides that are tethered to the particles, and the ends of the bridging moieties hybridize with the bridging moieties but not the unique particle identifier sequences. Further details of the probe system may be found in the methods section above. In some embodiments, the population of bridging moieties of (b) is a population of grid oligonucleotide molecules, wherein the sequence at the terminus at one end of the grid oligonucleotide molecules is complementary to a grid oligonucleotide binding sequence and the sequence at the terminus of other end of the grid oligonucleotide molecules is complementary to a grid oligonucleotide binding sequence. In these embodiments, the grid oligonucleotide molecules may be single molecules or split, and if the grid oligonucleotide molecules are split into one or more sequences then the system further comprises one or more splint oligonucleotides that hold the sequences together.

In some embodiments, the bridging moiety may be a particle that has surface tethered oligonucleotides, as illustrated in FIG. 22. As illustrated, in any embodiment the bridging moiety (e.g., a particle) may contain UMIs that are added to adjacent particles, which generates pairs UMIs that are in one molecules. The UMI pairs can be sequenced and decoded to identify which particles are in proximity.

In some embodiments, the population of barcoded particles may comprise a first set of barcoded particles and a second set of barcoded particles: wherein: i. the surface-tethered oligonucleotides of the first set of barcoded particles comprise a first bridging moiety binding sequence, and ii. the surface-tethered oligonucleotides of the second set of barcoded particles comprise a second bridging moiety binding sequence; and iii. in the population of bridging moieties of (b), the oligonucleotide sequences comprise a first sequence that is complementary to the first bridging moiety binding sequence and a second sequence that is complementary to the bridging moiety binding sequence. This implementation is shown in FIG. 3. In these embodiments, the first and second sets of particles may each comprise at least 10 members (e.g., at least 100, at least 1,000, at least 1,000, at least 10,000, at least 100,000, at least 1M at least 10 M, at least 100 M, at least 1 B or at least 10 B) members.

In some embodiments, the population of bridging moieties of (b) is a population of grid oligonucleotide molecules and the sequence at the terminus at one end of the grid oligonucleotide molecules may be complementary to the first grid oligonucleotide binding sequence and the sequence at the terminus of other end of the grid oligonucleotide molecules may be complementary to the second grid oligonucleotide binding sequence. In these embodiments, the grid oligonucleotide molecules are single molecules or split, and if the grid oligonucleotide molecules are split into one or more sequences then the system further comprises one or more splint oligonucleotides that hold the sequences together. In some embodiments, the bridging moiety binding sequences are adjacent to the unique particle identifier sequences in the surface-tethered oligonucleotides, and the ends of the oligonucleotide sequences of the bridging moiety hybridize with the bridging moiety binding sequences but not the unique particle identifier sequences.

Also provided is a population of barcoded particles that comprise surface-tethered oligonucleotides, wherein the surface-tethered oligonucleotides comprise have a unique particle identifier sequence and a bridging moiety binding sequence, wherein the population comprises a first set of barcoded particles and a second set of barcoded particles: wherein: i. the surface-tethered oligonucleotides of the first set of barcoded particles comprise a first bridging moiety binding sequence, and ii. the surface-tethered oligonucleotides of the second set of barcoded particles comprise a second bridging moiety binding sequence. In these embodiments, the bridging moiety binding sequences in the surface-tethered oligonucleotides may be adjacent to the unique particle identifier sequences. In any embodiment, the population of particles the first and second sets each comprise at least 10, least 100, at least 1,000, at least 1,000, at least 10,000, at least 100,000, at least 1M at least 10 M, at least 100 M, at least 1 B or at least 10 B members.

Also provided by this disclosure are kits for practicing the subject methods, as described above. In certain embodiments, the kit may comprise the components of the probe system or starting products for making the components. The kit may additionally contain a ligase, nucleotides, and/or a polymerase for a gap-fill/ligation, ligation or polymerization reactions. The various components of the kit may be present in separate containers or certain compatible components may be pre-combined into a single container, as desired. In addition to the above-mentioned components, the subject kit may further include instructions for using the components of the kit to practice the subject method.

### EXAMPLES

The following examples are put forth so as to provide those of ordinary skill in the art with additional disclosure and description of how to make and use the present invention, and are not intended to limit the scope of what the inventors regard as their invention nor are they intended to represent that the experiments below are all or the only experiments performed.

### Example 1

The following example provides a way to analyze proteins and/or RNA in or on single cells without the need to compartmentalize the single cells, or microscopy. The method can be used to analyze cells in suspension, e.g., immune cells isolated from a body fluid, blood or a tissue, or fixed tissues or tissue sections that have been immobilized on a surface (e.g., a glass slide, for example). Such methods have traditionally used microscopy to image the cells. Here, the microscopy is eliminated and, instead, a binding pattern can be analyzed by DNA sequencing. In this method, the spatial relationships between the barcoded particles are determined to provide a map (in which each barcoded particle can be considered a “pixel”) and the sites to which the capture agent binds are mapped to a barcoded particle. The method makes use of random bar-coded (also called a “unique particle identifier sequence” or unique molecular identifier or “UMI”) particles, which can have a defined diameter of a few tens of nm to a hundred nm. The present method does not rely on proximal diffusion; rather the present method relies on a bridging moiety that hybridize adjacent particles. In the following examples, the bridging moiety is an oligonucleotide and may be referred to as a “grid” oligonucleotide, where a grid oligonucleotide has a 3′ and a 5′ end and sequences that hybridize to the particles. Other types of bridging moiety may also be used, e.g., moieties that have multiple nucleic termini that can be extended.

In the examples shown below, target analyte proteins and/or RNA may be bound by either a protein-specific antibody linked to a DNA tag and/or RNA binding nucleic acid probe. Each analyte-specific probe type carries a unique fixed and known (not random) bar code for target identification.

### Example 2

The following description provides a way to analyze a suspension of cells, e.g., lymphocytes.

The average lymphocyte has a volume of 130 um\hat{ }3 and a surface area of about 124 um\hat{ }2. In this example, the average diameter of particle is around 50 nm. This exemplary lymphocyte could have several thousand particles covering it assuming a monolayer of particles on the surface of the cell.

$$\begin{matrix}V = 4 / 3 pi r \hat{} 3 \\ A = pi \hat{} 1 / 3 \times ( 6 V ) \hat{} 2 / 3\end{matrix}$$

Thus, a typical cell is estimated to bind to several thousand particles, depending on the size of the particles.

In this example, cells in suspension are analyzed also with a spatial resolution of target proteins on the surface of each single cell, possibly providing valuable diagnostic information. Such information is often called cell polarity and regulates many immune cell functions (Russel et al. Journal of Cell Science 2008 121:131-136 and Oliaro J. et al PNAS Dec. 5, 2006 103 (49) 18685-18690). Using presently available methods, the analysis of cell polarity requires microscopy to analyze the immune cells limiting the analytical throughput to a few cells and a few targets in just a few samples. The present method is capable of quantifying the abundance and relative positions of hundreds to thousands of cell surface markers on millions of immune cells. Cell polarity (namely the uneven distribution of cell surface proteins on a cell) regulates many important functions and is very difficult to analyze for many proteins on many cells. The polarization regulates not only cell migration but also immune cell activity for example antigen presentation and effector functions.

### Example 3

A first implementation of the method is shown in FIG. 8. In this implementation of the method (and potentially others) in order for the grid oligonucleotide (one of several types of bridging moiety that could be used) to bind to two adjacent particles (which are indicated as “BEAD PIXELS” in the figure), and not the same particle, the particles are manufactured as at least two different types, shown as type 1 and type 2, which differ in their grid oligonucleotide binding sequence (GOBS). These sequences are illustrated shown as GOBS1 and GOBS2. This design alleviates potential competitive hybridization reactions that may otherwise lower the efficiency of detection.

In this method, all the particles used in the method may be sequenced beforehand to identify which pairs of UMIs are in each particle. This can be done by sequencing the strands that are eluted from the particles in the during manufacture of the particles by emulsion PCR.

In this implementation of the method, the UMIs from the adjacent particles are added to the grid oligonucleotide by a gap-fill/ligation reaction to produce an extended grid oligonucleotide that has forward and reverse PCR primer sites at the ends. Likewise, the UMIs that are adjacent to the surface-tethered oligonucleotides that are conjugated to the antibodies are added via a gap-fill/ligation reaction to produce an extended antibody oligonucleotide that also has forward and reverse PCR primer sites at the ends. The extended grid oligonucleotides and the antibody oligonucleotide can then be amplified and sequenced.

Sequencing the extended molecules identifies particles that are proximal to one another and that have been hybridized to the same grid oligonucleotide molecule. This information can be used to build a relational map of particles. The UMIs copied onto the antibody-bound oligonucleotides can then be mapped onto the map.

### Example 4

A second implementation of the method is shown in FIG. 8. In this implementation of the method (and potentially others): 1) Target cells are bound by the proximity probes (antibodies coupled to oligonucleotides barcoded for target protein identity containing either free 3′ ends or free 5′ends) or nucleic acid probes capable of binding to specific RNA sequences; 2) barcoded particles of at least two types are added to the sample and allowed to hybridize to the ends of the proximity probes via the “proximity probe binding sequences” (PPBS); 3) the bridging moiety is added to the sample and allowed to hybridize to GOBS1 and GOBS2 of either particle type. Optional washing steps between step 1, 2, 3, can be carried out to remove unbound reagents. 5) Next, the method may comprise allowing a DNA polymerase and dNTPs to extend hybridized 3′-ends and a ligase to unite the sequence, thereby encoding a PCR amplicon with the UMIs from the particles to produce a target amplicon spanning from one proximity probe to another via two particles, which UMIs provides their relative location. As the product spans at least two particles, a grid of proximal RCPs can be obtained. 6) Next, the method comprises amplifying the united amplicon with PCR and then sequencing it to decode the image of which proteins are where.

As with example 3, in order for the grid-oligonucleotide to bind to two particles, and not the same particle, at least two particle types are manufactured that differ in their grid oligonucleotide binding sequence (GOBS1 and GOBS2). UMIs are encoded into from the particles into the extended grid oligonucleotide.

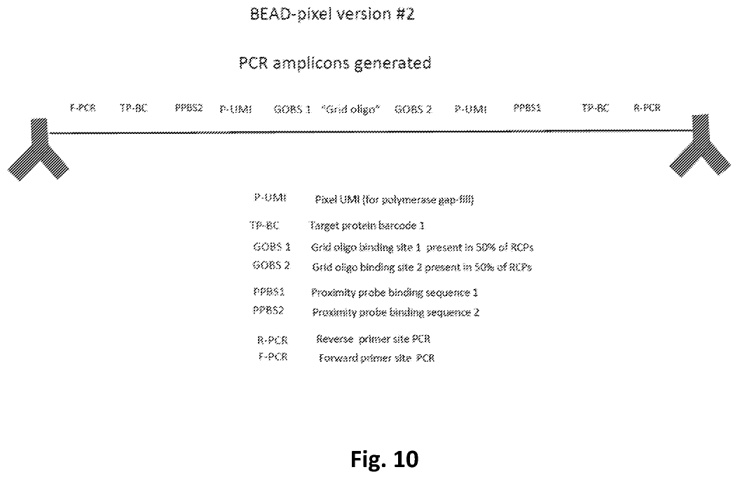

FIG. 10 schematically illustrates the structure of an extended grid oligonucleotide in this implementation of the method.

### Example 5

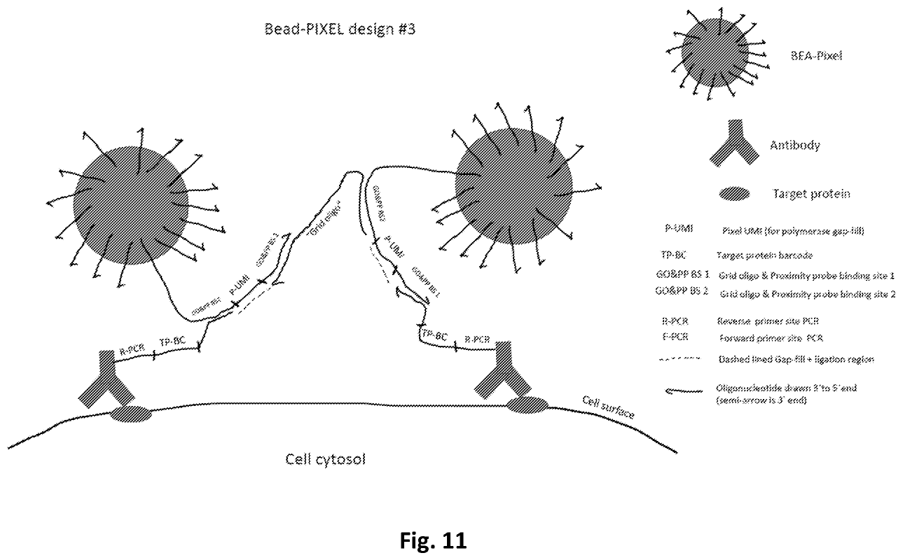

A third implementation of the method is shown in FIG. 11. In this implementation of the method: 1) the target cells are bound by the proximity probes (antibodies coupled to oligonucleotides barcoded for target protein identity containing either free 3′ ends or free 5′ends) or nucleic acid probes capable of binding to specific RNA sequences. 2) Pre-made particles of one type are added to the sample and allowed to hybridize to the ends of the proximity probes via the grid oligonucleotide and proximity probe binding sequences (GO&PP BS). As there is a surplus of GO&PP BS in the particles there will be binding sites left over for the oligonucleotide added in the next step; 3) The grid oligonucleotide is added to the sample and allowed to hybridize to GO&PP BS of the particles. Optional washing steps may be performed between step 1, 2, 3, in order to remove unbound reagents. The next step may comprise 5) Allowing a DNA polymerase and dNTPs to extend hybridized 3′-ends and a ligase to unite the sequence to thereby encode a PCR amplicon with the UMIs. This ligation produces a target amplicon spanning from one proximity probe to another via two particles, which provides their relative location. Because the product spans at least two particles a grid of proximal particles can be obtained. 6) Next the method involves amplifying the united amplicon by PCR and then sequencing it to decode the image of which proteins are where. Again, the UMIs are encoded into from particles into the extended grid oligonucleotide.

### Example 6

A fourth implementation of the method is shown in FIG. 12. In this implementation of the method: 1) the target cells are bound by the proximity probes (antibodies coupled to oligonucleotides barcoded for target protein identity containing either free 3′ ends or free 5′ends) or nucleic acid probes capable of binding to specific RNA sequences; 2) Pre-made barcoded particles of at least two types are added to the sample and allowed to hybridize to the ends of the proximity probes via the “proximity probe binding sequences” (PPBS); 3) the grid oligonucleotide is added to the sample and allowed to hybridize to GOBS1 and GOBS2 of either barcoded particle type. Optional washing steps may be performed between step 1, 2, 3, in order to remove unbound reagents; 5) next the method comprises allowing a DNA polymerase and dNTPs to extend hybridized 3′-ends and a ligase to unite the sequence to thereby encode a PCR amplicon with the UMIs from the adjacent barcoded particles, thereby forming a target amplicon spanning from one proximity probe to another via two barcoded particles to provide their relative location. As the product spans at least two particles a grid of proximal barcoded particles is obtained. 6) Next, the method comprises amplifying the united amplicon with PCR and then sequencing it to decode the image of which proteins are where. As with the examples above, in order for the grid oligonucleotide to bind to two proximal barcoded particles and not to the same barcoded particle, these barcoded particles are manufactured as at least two regions of differing sequences. In the embodiment shown, they differ in their GOBS sequence. Again, the UMIs are encoded from the barcoded particles into the extended grid oligonucleotide.

### Example 7

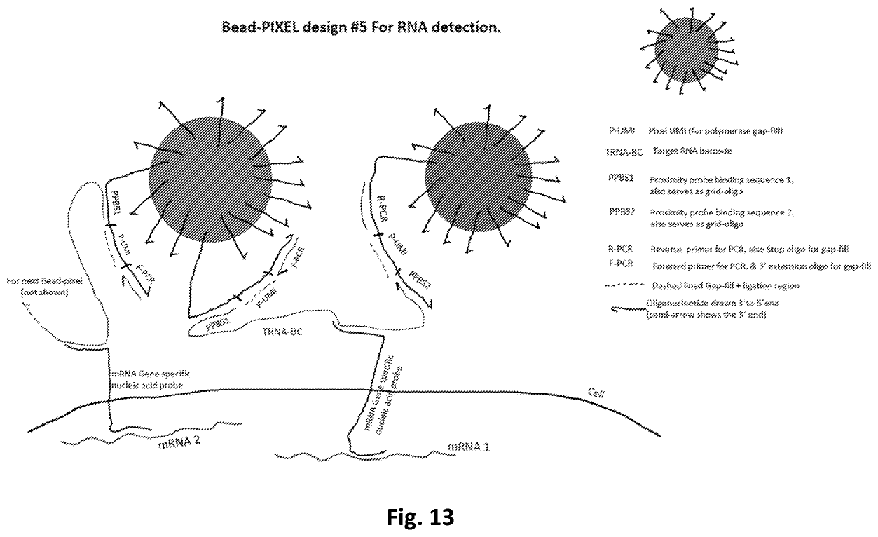

A fifth implementation of the method is shown in FIG. 13. In this implementation of the method, RNA is detected. As shown, the grid oligonucleotide is designed to bind to a site in a probe that hybridizes with a cellular RNA. In this implementation, the sequencing results should show that mRNA 1 is proximal to mRNA 2 in the cell (since the grid oligonucleotides that are bound to those mRNAs (indirectly, via the probes) both add the UMI for the left hand particle when they are extended in the gap-fill/ligation reaction.

### Example 8

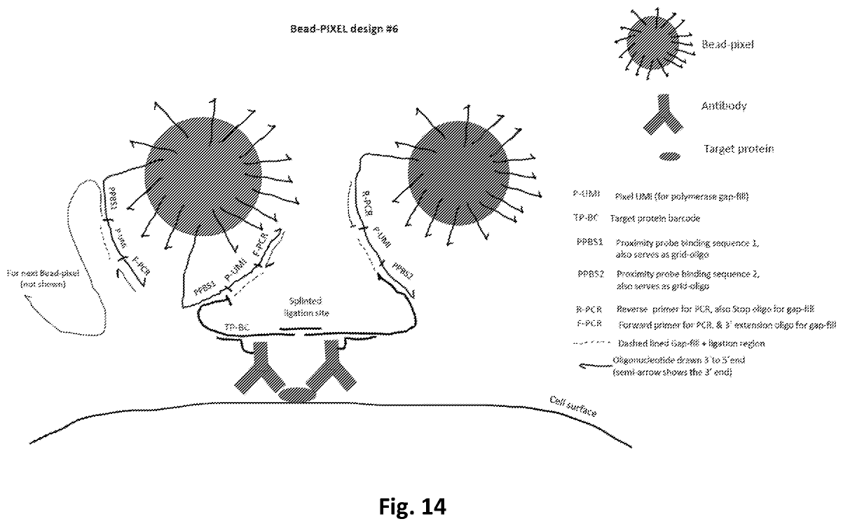

A sixth implementation of the method is shown in FIG. 14. In this implementation of the method, the grid oligonucleotide is split into two parts, which only join together when they are in close proximity to one another in the presence of a splint. The implementation shown in in FIG. 14 is similar to the implantation described in Example 6 above (and shown in FIG. 10) except that a splint-mediated proximity ligation assay (PLA) step ensures that the grid oligonucleotide will be a single molecule if two binding events to the same target molecule occur. This increases the specificity of the assay. In this example, the grid oligonucleotide is split into two and splinted together by a PLA splint. The PLA splint can be designed to only splint proximity probe pairs that target the same protein, which further increases specificity and multiplexability.

### Example 9

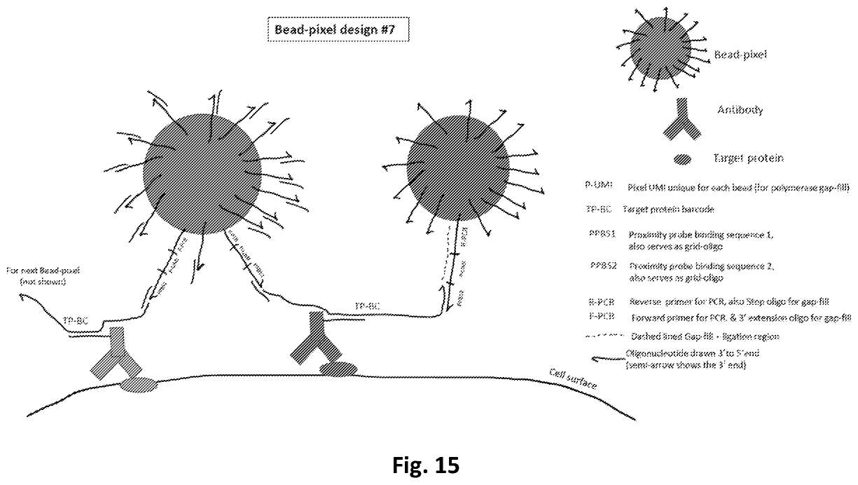

A seventh implementation of the method is shown in FIG. 15. This implementation utilizes the free 3′ ends of the particles to generate the PCR amplicon. In this example, the proximity probe (i.e., the oligonucleotide that is linked to the antibody) includes a sequence that is complementary to the grid oligonucleotide. For Type 1 particles (on the left) a splinted ligation reaction joins the P-UMI & F-PCR with the grid oligonucleotide and Type 2 particles (on the right) a polymerization reaction incorporates the P-UMI and the R-PCR. At least two different particle types are used in this implementation of the method.

### Example 10

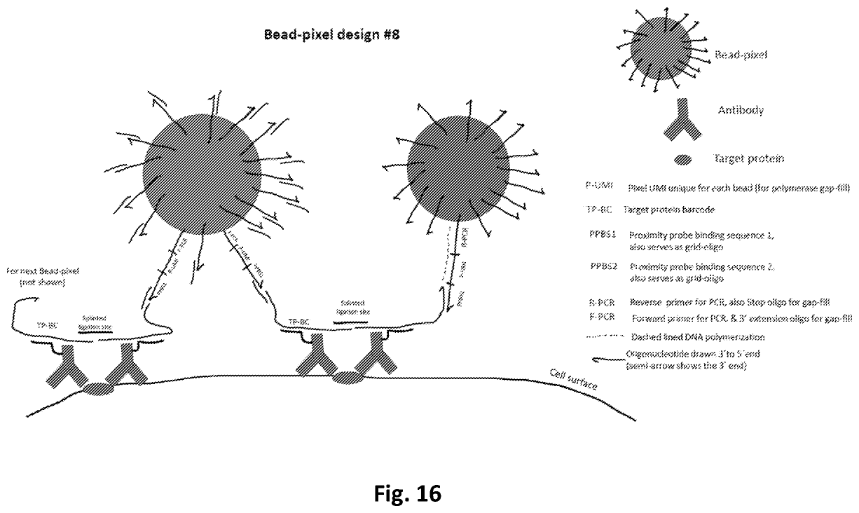

An eight implementation of the method is shown in FIG. 16. This implementation is essentially the same as design #7 described in example 9 above, but it includes a PLA step to increase specificity of the detected protein.

### Example 11

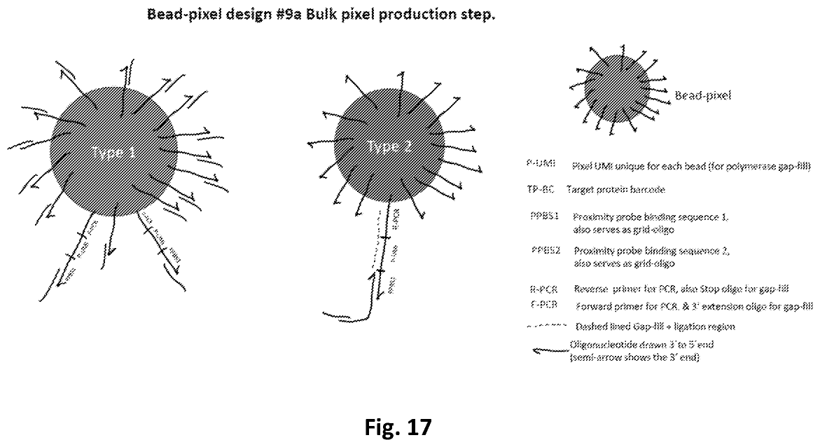

A ninth implementation of the method is shown in FIGS. 17 and 18. In FIG. 17, the barcoded particles are pre-hybridized with primers that extended before the particles are mixed with the sample. In this example, the particles are pre-prepared to enable a sample reaction that only requires a DNA ligation reaction, which simplifies the assay handling. Prior to the assay, in the manufacturing of the bead-pixels, the 3′ end of the oligonucleotides that are tethered to the type 1 particles (on the left) are hybridized with an oligonucleotide that acts as a splint for ligating the 5′ end of the grid oligonucleotide to the surface-tethered oligonucleotide. The 3′ end of this splint may be blocked so it cannot be extended. The type-2 particles (on the right) are pre-hybridized with an oligonucleotide that hybridizes to the 3′ end of the surface tethered oligonucleotide, which is extended by a polymerase to copy the P-UMIs and R-PCR sequences. As shown, this extended oligonucleotide has a splint that facilitates ligation to the grid oligonucleotide.

In this assay (as shown in FIG. 18) the sample is first bound by the target specific probes and then secondarily by the two types of particle shown in FIG. 17. A DNA ligation reaction joins the sequences of adjacent particles to the via the grid oligonucleotide that his hybridized to the target-specific probe. The entire sequence can then be amplified by PCR and sequenced to yield a map of pixels in proximity and the target biomolecules (proteins and or RNA) in the same vicinity, as described above.

### Example 12

A tenth implementation of the method is shown in FIG. 19. In this example, the surface tethered oligonucleotides from adjacent barcoded particles are ligated together using the bridging moiety as a splint. In this example, the ligation product can be readily amplified and sequenced using forward and reverse primers that are in the oligonucleotides that are ligated together. This implementation uses particles that have free 3′ ends and particles that have free 5′ ends. Free 5′ end particles can be made by pre-hybridizing an oligonucleotide to the surface tethered oligonucleotide and making a complement of the UMI by polymerization (not shown in figure). After binding of the target-specific probes (i.e., the antibodies) to the sample, the barcoded particles are added to the sample, where one type of particle may be pre-hybridized with the bridging moiety, which acts as a ligation splint, or the splint may be added later. A single ligation then ligates the particles together and ligates each target-specific oligonucleotide to a particle. The ligation products can be amplified PCR, sequenced, and analyzed to provide a map of barcoded particles as well as the targets that bind to those particles.

### Example 13

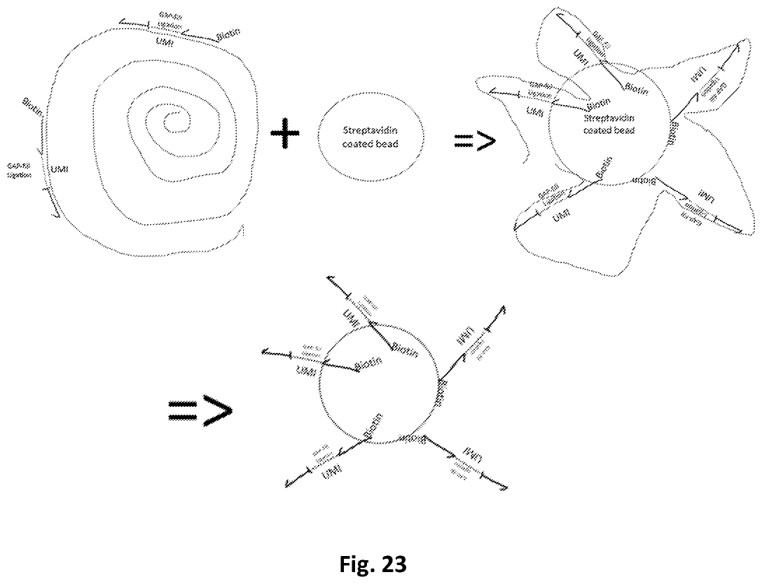

FIG. 23 schematically illustrates another way by which barcoded particles can be produced. In this example, barcoded RCA products (which each contain hundreds to thousands of repeat UMIs) are used as templates to copy the UMIs onto biotinylated primers by DNA polymerization reaction (e.g., a gap-fill/ligation or a primer extension reaction). Without denaturing the primer extension products from the RCA products, the RCA products (with the biotinylated extension products) are then allowed to bind to streptavidin coated beads. The biotin binds to the beads and because proximal UMIs (from the same RCA product) are bound to nearby sites on the streptavidin coated beads, the become barcoded with a cluster of UMIs. After the primer extension products are bound to the beads, the RCA products can be then removed by dehybridization (e.g., by heat, or a denaturant) or UNG degradation, revealing 3′ free UMI barcoded beads.

1. A method for making a physical map of a population of barcoded particles, comprising:
(a) producing a complex comprising:
i. a population of barcoded particles, wherein the barcoded particles are uniquely barcoded by surface-tethered oligonucleotides that have unique particle identifier sequences; and
ii. a population of bridging moieties that comprises oligonucleotide sequences;
wherein the bridging moieties hybridize directly or indirectly via a splint to complementary sites in the surface-tethered oligonucleotides;
(b) performing a ligation, polymerization, and/or a gap-fill/ligation reaction on the complex, thereby producing reaction products that comprise pairs of unique particle identifier sequences or from adjacent barcoded particles, or complements thereof;
(c) sequencing the reaction products produced in step (b);
(d) analyzing the sequences to identify which pairs of unique particle identifier sequences or complements thereof have been copied and/or ligated together in step (b); and
(e) making one or more physical maps of the barcoded particles using the pairs of sequences identified in (d).

2. The method of claim 1, wherein step (b) is done by ligation.

3. The method of claim 1, wherein step (b) is done by a polymerization or gap-fill/ligation reaction.

4. The method of claim 1 or 2, wherein, in step (a) the bridging moieties splint the surface-tethered oligonucleotides from two adjacent barcoded particles together and wherein:
step (b) comprises performing a ligation on the complex, thereby producing reaction products that comprise pairs of unique particle identifier sequences from adjacent barcoded particles; and
step (c) comprises sequencing the reaction products produced in step (b).

5. The method of any claims 1-3, wherein the method comprises: (b) extending the bridging moieties that are hybridized to surface-tethered oligonucleotides of two barcoded particles to add the unique particle identifier sequences from the two barcoded particles or their complements to the bridging moieties;
(c) sequencing the extended bridging moieties;
(d) analyzing the sequences to identify which pairs of unique particle identifier sequences or complements thereof have been added onto the bridging moieties; and
(e) making one or more physical maps of the barcoded particles using the pairs of sequences identified in (d).

6. The method of any prior claim, wherein step (a) comprises:
hybridizing the population of bridging moieties and the population of barcoded particles, wherein either the bridging moieties or the barcoded particles are immobilized, and wherein:
(i) the surface-tethered oligonucleotides of the barcoded particles each have a bridging moiety binding sequence in addition to a unique particle identifier sequence, and
(ii) the bridging moieties each comprise a first terminal sequence that is complementary to a bridging moiety binding sequence and a second terminal sequence that is complementary to a bridging moiety binding sequence; and
(iii) at least some of the bridging moieties hybridize to surface-tethered oligonucleotides two adjacent barcoded particles.

7. The method of any prior claim, wherein the extending comprises a polymerization, and/or, gap fill and/or ligation reaction, which adds the unique particle identifier sequences from the two adjacent barcoded particles, or their complements, onto the bridging moiety.

8. The method of any prior claim, wherein in step (a):
(i) the population of barcoded particles comprises a first set of barcoded particles and a second set of barcoded particles, wherein:
i. the surface-tethered oligonucleotides of the first set of barcoded particles further comprise a first bridging moiety binding sequence, and
ii the surface-tethered oligonucleotides of the second set of barcoded particles further comprise a second bridging moiety binding sequence;
(ii) the bridging moieties each comprise a first terminal sequence that is complementary to the first bridging moiety binding sequence and a second terminal sequence that is complementary to the second bridging moiety binding sequence; and
(iii) at least some of the bridging moieties hybridize to surface-tethered oligonucleotides two adjacent barcoded particles.

9. The method of any prior claim wherein the products of step (b) are amplified by PCR prior to sequencing.

10. The method of any prior claim, wherein the bridging moieties are immobilized and the barcoded particles are hybridized to the immobilized bridging moieties molecules.

11. The method of claim 10, wherein the bridging moieties are hybridized to sequences that are in or on a cell, prior to hybridization with the barcoded particles.

12. The method of claim 10, wherein the bridging moieties are made in situ in or on a cell, prior to hybridization with the barcoded particles.

13. The method of any prior claim, wherein the barcoded particles are immobilized and the bridging moieties are hybridized to the immobilized barcoded particles.

14. The method of claim 13, wherein the barcoded particles are hybridized to sequences that are in or on a cell, prior to hybridization with the bridging moieties.

15. The method of any prior claim, wherein the bridging moieties or the barcoded particles are immobilized via an antibody.

16. The method of any prior claim, wherein the bridging moieties or the barcoded particles are immobilized via a nucleic acid probe.

17. The method of any prior claim, wherein the bridging moieties or barcoded particles are immobilized on one or more surfaces

18. The method of any prior claim, wherein the bridging moieties or barcoded particles are immobilized to sites that are in or on one or more cells, wherein the cells are in suspension or attached to a support.

19. The method of claim 18, wherein the bridging moieties or the barcoded particles are immobilized to sites that are in or on one or more cells via one or more binding agents, wherein the binding agents are each bound to a sequence in bridging moiety or barcoded particle and a site in or on the one or more cells.

20. The method of claim 19, further comprising performing a proximity assay between one or more binding agents and the barcoded particle to which they are bound.

21. The method of claim 20, wherein the proximity assay produces assay products that contains a binding agent identifier sequence or complement thereof and a unique particle identifier sequence or complement thereof.

22. The method of claim 21, wherein the method comprises:
mapping the binding agents to the physical map of the immobilized particles by analyzing which unique particle identifier sequences and which binding agent identifier sequences are in the assay products.

23. The method of claim 22, wherein the binding agent identifier sequence or complement thereof and the unique particle identifier sequence or complement thereof are incorporated into the extended bridging moieties of step (b).

24. The method of any prior claim, wherein the bridging moieties uniquely hybridizes to a binding agent identifier sequence.

25. The method of any prior claim, wherein bridging moieties are not rolling circle amplification (RCA) products.

26. The method of any prior claim, wherein the bridging moieties are grid oligonucleotides.

27. The method of any prior claim, wherein the bridging moieties are particles that have surface-tethered oligonucleotides.

28. A probe system comprising:
(a) a population of barcoded particles, wherein the barcoded particles are uniquely barcoded by surface-tethered oligonucleotides that have a unique particle identifier sequence and a bridging moiety binding sequence; and
(b) a population of bridging moieties that comprises oligonucleotide sequences, wherein the oligonucleotide sequences are complementary to the bridging moiety binding sequence of the surface-tethered oligonucleotides,
wherein hybridization of (a) and (b) produces a complex in which the bridging moieties hybridize to adjacent barcoded particles.

29. The probe system of claim 28, wherein the population of bridging moieties of (b) is a population of grid oligonucleotide molecules, wherein the sequence at the terminus at one end of the grid oligonucleotide molecules is complementary to a grid oligonucleotide binding sequence and the sequence at the terminus of other end of the grid oligonucleotide molecules is complementary to a grid oligonucleotide binding sequence,

30. The probe system of claim 29, wherein the grid oligonucleotide molecules are single molecules or split, and if the grid oligonucleotide molecules are split into one or more sequences then the system further comprises one or more splint oligonucleotides that hold the sequences together;

31. The probe system of any of claims 28-30, wherein the bridging moieties act as a splint so that surface-tethered oligonucleotides from different particles can be ligated together.

32. The probe system of any of claims 27-31, wherein:
the population of barcoded particles of (a) comprises a first set of barcoded particles and a second set of barcoded particles: wherein:
i. the surface-tethered oligonucleotides of the first set of barcoded particles comprise a first bridging moiety binding sequence, and
ii the surface-tethered oligonucleotides of the second set of barcoded particles comprise a second bridging moiety binding sequence; and
iii. in the population of bridging moieties of (b), the oligonucleotide sequences comprise a first sequence that is complementary to the first bridging moiety binding sequence and a second sequence that is complementary to the bridging moiety binding sequence

33. The probe system of claim 32, wherein the population of bridging moieties of (b) is a population of grid oligonucleotide molecules and, the sequence at the terminus at one end of the grid oligonucleotide molecules is complementary to the first grid oligonucleotide binding sequence and the sequence at the terminus of other end of the grid oligonucleotide molecules is complementary to the second grid oligonucleotide binding sequence.

34. The probe system of claim 33, wherein the grid oligonucleotide molecules are single molecules or split, and if the grid oligonucleotide molecules are split into one or more sequences then the system further comprises one or more splint oligonucleotides that hold the sequences together.

35. The probe system of any of claims 28-34, wherein the first and second sets of barcoded particles each comprise at least 10 members.

36. The probe system of any of claims 28-35, wherein the bridging moiety binding sequences are adjacent to the unique particle identifier sequences in the surface-tethered oligonucleotides, and the ends of the oligonucleotide sequences of the bridging moiety hybridize with the bridging moiety binding sequences but not the unique particle identifier sequences.

37. The probe system of any of claim 28, 31, 34, 35 or 36, wherein the bridging moieties are particles that have surface-tethered oligonucleotides.

38. A population of barcoded particles that are uniquely barcoded by surface-tethered oligonucleotides that have a unique particle identifier sequence and a bridging moiety binding sequence, wherein the population comprises a first set of barcoded particles and a second set of barcoded particles: wherein:
i. the surface-tethered oligonucleotides of the first set of barcoded particles comprise a first bridging moiety binding sequence, and
ii the surface-tethered oligonucleotides of the second set of barcoded particles comprise a second bridging moiety binding sequence.

39. The population of barcoded particles of claim 38, wherein the bridging moiety binding sequences in the surface-tethered oligonucleotides are adjacent to the unique particle identifier sequences.

40. The population of barcoded particles of claim 38 or 39, wherein the first and second sets of barcoded particles each comprise at least 10 members.

41. A method for manufacturing barcoded particles, comprising:
(a) hybridizing a population of RCA products that have unique identifier sequences with a population of particles that have surface-tethered oligonucleotides, wherein a plurality of the particles hybridize to a single RCA product and multiple surface-tethered oligonucleotides of each of the plurality of the particles hybridize to a site in the RCA product that is upstream of the identifier sequences, and
(b) (i) cleaving the RCA products at a site that is downstream of the identifier sequences in the RCA products, or (ii) extending the surface tethered oligonucleotides using the hybridized RCA products as a template, thereby adding identifier sequences from the RCA products to the particles.

42. A method for manufacturing barcoded particles, comprising:
(a) hybridizing a population of RCA products that have unique identifier sequences with biotinylated primers that hybridize to sites that are upstream of the unique identifier sequences;
(b) extending the primers to produce multiple biotinylated primer extension products per RCA product, using polymerization (e.g., gap-fill/ligation or primer extension) reaction;
(c) without denaturing the biotinylated primer extension products from the RCA product to which they are annealed, mixing the RCA products and hybridized primer extension products to a streptavidinated particles such that at last several RCA product binds to a single particle, thereby binding the hybridized primer extension products to the particles, and
(d) removing the RCA products, thereby leaving the biotinylated primer extension products on the particles.
